(19) United States
(12) Patent Application Publication
(10) Pub. No.: US 2004/0063612 A1
(43) Pub. Date: Apr. 1, 2004

(54) Neuroprotective agents

(76) Inventor: Manssur Yalpani, Rancho Santa Fe, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/672,257

(22) Filed: Sep. 26, 2003

Related U.S. Application Data
(60) Provisional application No. 60/414,694, filed on Sep. 26, 2002.

(51) Int. Cl. A61K 38/00; C07K 14/00

(52) U.S. Cl. 514/2; 530/300

(57) ABSTRACT

The present invention relates to methods of treating neurodegenerative diseases with the neuroprotective agents of Formulas I-IV and XII and the other compounds described herein. The neuroprotective agents inhibit nitric oxide synthase enzymes and in particular nitric oxide synthase III (NOS III) and can be used to treat Alzheimer's disease.

### CROSS-REFERENCE TO RELATED APPLICATIONS

This application claims the benefit of U.S. Provisional application No. 60/414,694, filed Sep. 26, 2002.

### FIELD OF THE INVENTION

The present invention relates to neuroprotective compounds, methods for preventing and treating neurodegenerative diseases and therapeutic compositions that contain said compounds, derivatives or pharmaceutically acceptable salts as active ingredients. The neuroprotective compounds of the present invention inhibit nitric oxide synthases and exhibit a neuroprotective activity and are useful as therapeutics of neurological diseases.

### BACKGROUND OF THE INVENTION

Apoptosis, or programmed cell death may arise from stroke, heart attack or other brain or spinal chord ischemia or trauma. As an integral part of the aging and development of the central nervous system (CNS), apoptosis is linked to the pathogenesis of neurodegenerative diseases, such as Alzheimer's disease, Parkinson's disease (PD), Pick's disease and Creutzfield-Jacob disease (Esiri M., Morris J. E. The Neuropathology of Dementia, 1997).

Apoptosis involves nuclear and cytoplasmic shrinkage and DNA fragmentation. Cysteine proteases known as caspases trigger DNA degradation. Nitric oxide synthases (NOSs) oxidize of L-arginine to citrulline, generating nitric oxide (NO), which is implicated in apoptosis. Of the three known NOSs, two (NOS I and NOS III) synthesize NO in CNS neurons. Elevated NO levels have both beneficial and detrimental effects on CNS cell viability and function. NO's detrimental effects result from its reaction with superoxide anion that generates the oxidant peroxynitrite (ONOO), which causes DNA strand breaks. These events can be abrogated by NOS inhibition, free radical scavengers, growth factor repletion following injury or genetic depletion of the NOS I gene. Brains of AD patients display abnormal NOS III gene expression and apoptosis likely represents an important pathogenic mechanism of neurodegeneration. Neuronal loss and synaptic disconnection cause cognitive impairment in AD and other neurodegenerative disorders.

Alzheimer's disease (AD) is a devastating, still incurable disorder with progressive dementia as its hallmark. To date, the only treatment for ischemic stroke targets clots in blocked blood vessels and is suitable for only a very small number of patients. No approved treatment exists for the remaining patients suffering the most severe strokes.

Early neurodegenerative disease studies focused on the impaired presynaptic cholinergic function. Many neurotransmitter systems are affected in AD. However, degeneration in the cholinergic system occurs earlier than in other systems. All available AD medications (Tacrine, Donepezil, Rivastigmine, and Galantamine) are cholinesterase inhibitors (ChEIs). They modestly slow cognitive decline and require lifelong treatment. Only half the patients treated with any one of these therapeutics actually derive any benefits and up to 40% of patients suffer side effects.

Other therapeutic targets are being examined, including calcium and potassium channel blockers (Nakagawa et al., Neurosci. Lett., 320, 33-6, 2002). Certain natural neuroprotective agents may protect neurons from apoptosis. Since immune and inflammatory reactions occur in the brains of AD patients, therapeutic efforts aimed at these mechanisms may be beneficial (Markesbery, et al., Brain Pathol., 9, 133-46, 1999). Cyclooxygenase (COX) inhibitors are among the anti-inflammatory developmental drugs (Bredt D., Snyder S., Neuron, 13, 301-313, 1994).

Other efforts target potential neuroprotective agents, such as amino acid and peptide derivatives (Findeis M A, Curr. Top. Med. Chem., 2, 417-23, 2002), e.g., the tripeptide pGlu-Glu-Pro-NH₂ (Koenig et al., Peptides, 22, 2091-7, 2001), D-cycloserine (Tsai et al., Am. J. Psychiatry, 156, 467-9, 1999), activity-dependent neurotrophic factor, Posatirelin (Zamostiano et al., Neurosci. Lett., 264, 9-12, 1999), stearyl-Lys-Lys-Tyr-Leu-NH₂ (Gozes et al Proc. Natl. Acad. Sci. USA, 96, 4143-8, 1999), and Huperzine A (Ved et al., Neuroreport, 8, 963-8, 1997). Proposed NOS inhibitors include 2-imino-4-methylpiperidine (Webber et al., J. Med. Chem., 41, 96-101, 1998), N6-(1-iminoethyl)-L-lysine (Stenger et al., Eur. J. Pharm., 294, 703-712, 1995) and arginine analogues, e.g., monomethyl-L-arginine (Southan, G. J., Szabo, C., Biochem. Pharm., 51, 383-394, 1996). Certain poly(amino acids) are reported to stimulate outgrowth of cultured neurites and induce the formation of neuronal networks (Hefti et al., Brain Res., 541, 273-83, 1991). Administration of general NOS inhibitors is impractical and possibly dangerous, since NO has important effects on cerebral circulation. While high NOS levels may be toxic to some neurons, other neurons require it for survival.

Various schemes have been proposed to block glutamate stimulation. PD patients treated with Amantadine survive longer compared to those without. Gabapentin and riluzole have purported efficacy in motor neuron disease. The use of branched chain amino acids (L-leucine, L-isoleucine, and L-valine) in ALS was proposed as these amino acids inhibit glutamate production.

There is mounting evidence of the specific neurological benefits of catechins, flavenoids, ginko, hydroxycinnamic acids, polyphenols, and vitamins. Some clinical data already supports therapeutic strategies involving oxidative stress reduction: interventions with vitamins E and C, L-deprenyl (Parkinson Study Group, N. Eng. J. Med., 328, 176-183, 1993) provided limited benefit in delaying conventional dopamine treatments in PD patients (Fahn, H., Am. J. Clin. Nutr., 53, 380S-382S, 1991). Vitamin interventions have been proposed (Sano et al., N. Engl. J. Med., 336, 1216-1222, 1997), as some cognitive performance improvements in AD patients are achievable with vitamins A and B complex (Franchi et al., Arch. Geront. Geriatrics, 6, 207-214, 1998). Agents such as N-acetyl cysteine, nitro-D-arginine methyl ester, L-arginine, or arginine-pyroglutamate can affect a partial rescue from neuronal apoptosis. Other proposed neuroprotectants include aromatic trifluoromethyl ketone derivatives (Kawase et al., Biol. Pharm. Bull., 24,1335-7, 2001) and fructose-1,6-bisphosphate (Donohoe et al., Brain Res., 917, 158-66, 2001).

Several recent studies implicate the role of metals in AD (Curtain et al., J. Biol. Chem., 276, 20466-73, 2001). Amyloid β-peptide (Aβ) is the major constituent of extracellular plaques and amyloid deposits. Other metals, however, may offer neuroprotection in dementia and ischaemic stroke. Thus, magnesium exhibits a range of neuronal activities that may ameliorate ischaemic CNS insults, including stroke (Muir K W., CNS Drugs, 15, 921-30, 2001). Significant neuroprotection with Mg is observed in cerebral ischaemia, with infarct volume reductions of 25-61%. Lithium (De-Maw Chuang, Ann. N.Y. Acad. Sci. 2001, 939, 404) and copper (Alcaraz-Zubeldia et al., Neurochem. Res., 26, 59-64 2001) may also be neuroprotective. However, there is no fundamental knowledge as yet on the neuroprotective activity of metals in general and their safe administration to patients suffering from neurodegenerative diseases.

The prior art does not describe the role of arabinogalactans, polyglutamic acid or poly(amino acids) in general as neuroprotective agents. Polyglutamylation is, of course, well known as an important posttranslational modification of neuronal α-tubulin.

Other approaches for neuroprotective agents include therapies aimed at decreasing β-amyloid production or deposition, immunizing against β-amyloid accumulation, and utilizing nerve growth factor to preserve the viability of vulnerable cholinergic neurons. Neither of these strategies is yet in large-scale clinical trials.

Despite all these efforts, no firm prospects exist for preventing neurodegenerative diseases. New therapeutic agents are therefore urgently needed to protect against premature cell death and restore or prolong the function of surviving damaged neurons in stroke, heart attack, brain or spinal cord trauma, and in neurodegenerative disorders.

### SUMMARY OF THE INVENTION

Briefly, in accordance with the present invention, the neuroprotective compounds of the present invention (Formulas I-IV, XII and other compounds described herein, namely the tripeptides and the compounds listed on pages 21 and 22 hereinafter referred to as “other compounds”) are administered to mammals in amounts effective to inhibit nitric oxide synthase enzymes (NOS) and in particular NOS III. The inhibition of the NOS provides a method of preventing, slowing the progression of and/or treating neurodegenerative diseases. Additionally, the present invention relates to novel polyglutamate polymer compositions having a polydispersity (P_{D}=M_{w}/Mₙ) of less than about 1.3.

Of particular interest in practicing the present invention a gamma-polyglutamate (γ-PGA) is employed in a method to inhibit NOS in a mammal. The molecular weight of the γ-PGA is from about 100,000 to about 7,000,000 Daltons and is usually from about 250,000 to about 2,000,000. Preferably, the molecular weight of the γ-PGA is about 1,000,000 Daltons. The γ-PGA can be made by the fermentation of Bacillus licheniformis and fractionated to make a monodisperse γ-PGA. The monodisperse γ-PGA is used to inhibit NOS III and treat neurodegenerative diseases such as Alzheimer's disease and stroke.

The present invention relates to the neuroprotective compounds (Formulas I-IV and XII and other compounds described herein) and methods for preventing, slowing the progression of and treating neurodegenerative diseases, cerebral infarction [cerebral hemorrhage, subarachnoid hemorrhage, cerebral infarction (atherothrombotic infarction, lacunar infarction and cardiogenic embolism)], transient ischemic attack and cerebral edema, traumatic brain injury, spinal injury, pain [headache (migraine, tension headache, cluster headache and chronic paroxysmal headache)], Parkinson's disease, Alzheimer's disease, seizure, disorders of the central nervous system, mood and emotional disorders, anxiety and psychosis, substitution therapy, morphine tolerance or dependence; thyroid disease, neuroendocrine disorders and dysregulation of food intake, disorders of nociception and pain control; autonomic disorders, cardiovascular dysfunction, co-medication in surgical procedures; septic shock, chronic rheumatoid arthritis, osteoarthritis, viral or non-viral infections and diabetes mellitus. The invention further relates to pharmaceutical compositions that contain said compounds or pharmaceutically acceptable salts as active ingredients. Additionally, PGA can be employed as a laxative.

The neuroprotective compounds of the present invention include compounds of general formulas I to II below:

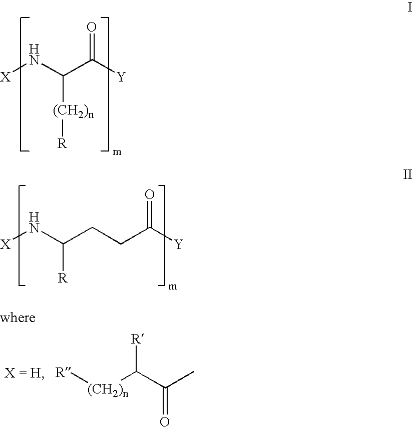

and for formula I:

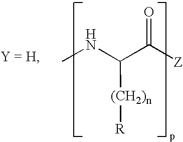

and for formula II:

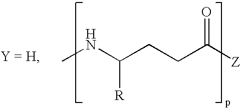

and for formulas I and II:

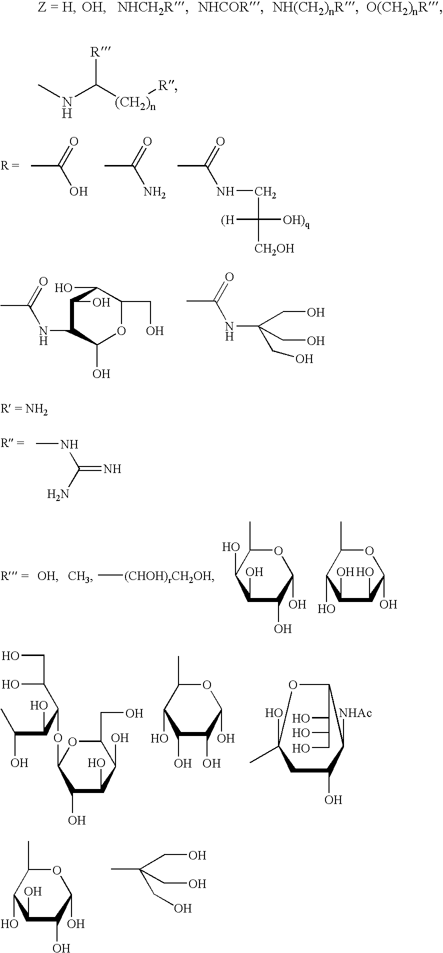

wherein m=1-70,000, preferably 20-50,000, and most preferably 3,000-10,000; n=1-5, p=0-3, q=1-6, and r=1-6. R may optionally also represent the metal salts of carboxylic acids when R=COOH where the metal is an essential metal such as aluminum, calcium, iron, lithium, manganese, magnesium, copper, selenium, and zirconium.

The neuroprotective compounds of the present invention furthermore include arabinogalactan compounds of general formula III below:

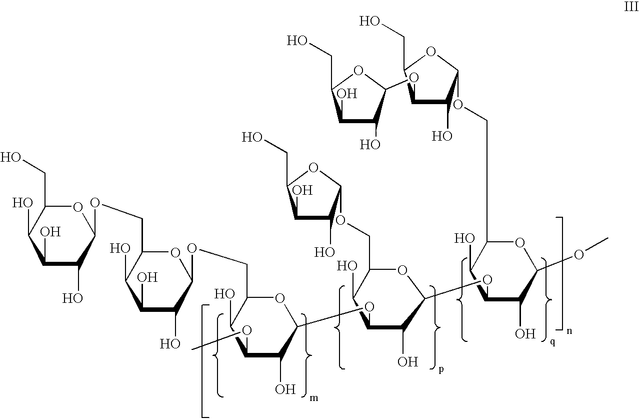

Arabinogalactans of formula III are derived from larch and comprise predominantly a 1,3-β-D-galactan backbone with 1,6-β-D-galactobiose, 1,3-β-L-arabinofuranosyl-α-L-arabinofuranose, and α-L-arabinofuranose branch units, with molecular weights of from about 6,000 to about 2,500,000, advantageously from about 50,000 to about 500,000 and preferably about 100,000 Da and L-arabinose:D-galactose ratios of between about 1.2:2.2, and preferably between about 1.6 to 1.7, and m>>q>p and m=1-300, preferably 50-250, and most preferably 100-200; q=1-50, preferably 10-40, and most preferably 20-30; p=1-20, preferably 1-10, and most preferably 3-5. Other arabinogalactans encompassed by this invention are derived from acacia (gum arabic), and constitute complex, branched arabic acid derivatives (molecular weight from about 10,000 to about 2,000,000, preferably from about 100,000 to about 500,000 and most preferably about 250,000 Da) with a 1,3-β-D-galactose backbone with O-6 linked branches composed of D-galactose, L-arabinofuranose and D-glucuronic acid units bearing additional L-rhamnose branches.

The novel neuroprotective compounds of the present invention furthermore include compounds of general formulas IV and XII below:

Y—(X)ₘ—R (IV)

Where Y=H, (CH₂)ₙ, O(CH₂)ₙ, [(CH₂)₂O]ₙ, N(CH₂)ₙ, alkyl, amino acid, or carbohydrate, (trishydroxymethyl)aminomethane and where n=1-6, and R=H, alkyl, amino acid, carbohydrate, fluoro- or halogen-substituent, (trishydroxymethyl)aminomethane, polyol or acyl residue, and where X comprises benzene, benzidine, S(−)-benzoin, benzophenone, diphenylamine, benzopinacole, cycloheptyl, cyclohexyl, cyclooctyl, cyclopentyl, furan, imidazole, iminostilbine, indazole, indole, indoline, bis(4-aminophenyl-1,4-diisopropylbenzene), phenyl, piperidine, pyridine, pyrrolidone, pyrrole, triazine, triphenylmethane, tryptamine, alkyl-, aryl, fluoro- and halogen-substituted derivatives thereof, or other aromatic, or cycloaliphatic residues, or heteroatom-containing cyclic residues comprising general formulae V-XI and substituted derivatives thereof, where m=1-4, and the bridging group, X, is:

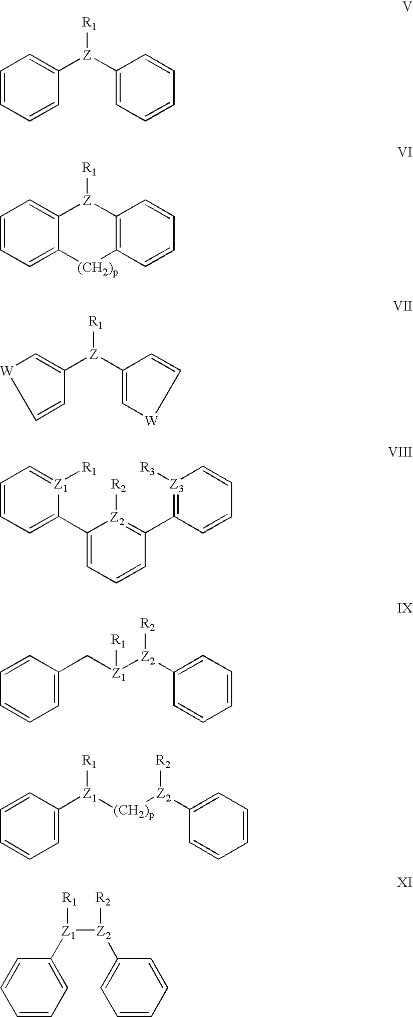

where

Z, Z₁, Z₂, Z₃=(CH₂)ₚ, C═O, CHO, N, NH, C═CHR₁, CH—NH, C═NNH, O, S

R₁, R₂, R₃=(CH₂)ₖR₄, O(CH₂)ₖR₄, OH, carbohydrate, amino acid, (trishydroxymethyl)aminomethane,

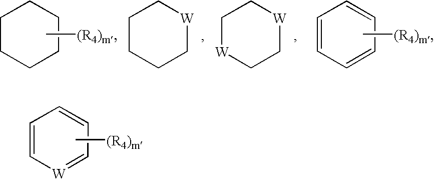

R₄=H, alkyl, fluorine, halogen, N-alkyl, (trishydroxymethyl)aminomethane

W=CH₂, CH₂R₅, CH, CHR₅, N(R₅)ⱼ, N, O, S, C═O

R₅=H, F, OH, alkyl, aryl and

where j=1-2, k=1-6, m′=1-5, p=1-10, q=1-6.

The novel neuroprotective compounds of the present invention furthermore include polymeric metal complexes of general formulas XII below:

P—[M] (XII)

Where P is an anionic polymer, including a polysaccharide, oligosaccharide, polypeptide, poly(amino acid), or synthetic polymer; and M is a metal ion that plays a neuroprotective role, such as aluminum, calcium, copper, lithium, magnesium, manganese, selenium, iron and zirconium. The compounds of Formula XII include both salts and complexes. Representative examples of polymeric metal complexes include polysaccharide iron complexes and poly(glutamic acid) iron complexes where the poly(amino acid) includes compounds of general formulas I and II of this invention, and the oligosaccharide and polysaccharide may be derived from alginate, arabinogalactan, cellulosics, e.g., carboxymethyl cellulose, dextran, galactomannan, konjac, maltodextrin, pectin, starch, starch glycolic acid, oxyamylose, oxycellulose, chondroitin sulfate, dextran sulfate, dextran phosphate, gellan and xanthan, and certain polynucleotides, e.g., polycyticyclic acid, polyinosinic acid, polyxenyl phosphate and where the synthetic polymer contains carboxylic acid, phosphate or sulfate functions, e.g., polyvinylsulfate, polyethylene sulfonate and polystyrene sulfonic acid, and polymers with repeating units of acrylic acid, methacrylic acid, fumaric acid, maleic acid, maleic anhydride, itaconic acid, crotonic acid, and the like. Polymers based on acrylic acid, maleic anhydride and methacrylic acid are particularly preferred, as are cross-linked polyacrylic acid, poly(acrylic acid-isobutyl vinyl ether and copolymers of acrylic acid and dimethylaminoethylacrylate). For P the molecular weight ranges from 1,000 to 10 million, preferably from 10,000 to 5 million, and most preferably 40,000 to 1 million Daltons.

The novel neuroprotective compounds of the present invention furthermore include additional compounds as described herein (in addition to the compounds of Formulas I-IV and XII) and extracts and actives derived from extracts of certain natural products, such as cranberry, blueberry, red sour cherry, tea and elderberry.

### BRIEF DESCRIPTION OF THE DRAWINGS

FIG. 1 shows the structures of α-PGA and γ-PGA.

FIG. 2 is an SEC/MALLS chromatogram of a commercial sample of α-PGA (obtained with 13.24 mg α-PGA, a Waters Ultrahydrogel 250 column and with 0.1 M citrate buffer (pH 2.0) as eluent).

FIG. 3 is a 3-dimensional SEC/MALLS chromatogram of the high molecular weight γ-PGA prepared in Example 1.

FIG. 4 show an SEC elution peak of fractionated γ-PGA from the MALLS detector II. The narrow peak distribution is indicative of a monodisperse material. Analysis confirmed that the P_{D} of the material was 1.0 with a molecular weight of 300,000 Da.

FIG. 5 is a graph showing the change in neuronal viability as a function of γ-PGA concentration.

FIG. 6 is a graph showing the change in neuronal viability as a function of arabinogalactan concentration.

FIG. 7 is a graph showing the change in neuronal viability as a function of γ-PGA concentration.

FIG. 8 is a graph showing the change in neuronal viability as a function of arabinogalactan concentration.

FIG. 9 is a graph showing the change in neuronal viability as a function of diphenylamine concentration.

FIG. 10 is a graph showing the change in mitochondrial function as a function of increasing multiplicity of adenovirus infections produced by a treatment with γ-PGA.

FIG. 11 is a graph showing the change in mitochondrial function as a function of increasing multiplicity of adenovirus infections (MOI) by a treatment with dichlorobenzhydrol phenoxyamine.

FIG. 12 is a graph showing the change in mitochondrial function as a function of increasing multiplicity of adenovirus infections (MOI) by a treatment with arabinogalactan.

### DETAILED DESCRIPTION OF THE INVENTION

In practicing the present invention one or more compounds of Formulas I-IV, XII and/or the other compounds described herein are administered to mammals to treat the neurodegenerative disorders described herein. The compounds are administered in an amount effective to inhibit one or more nitric oxide synthase enzymes in the mammal. Pharmaceutically acceptable salts and esters of the neuroprotective compounds are also employed in practicing the present invention.

In a preferred embodiment of the present invention one or more compounds of Formulas I-IV, XII and/or the other compounds described herein are administered to a human at a high risk of developing Alzheimer's disease to prevent or slow down its onset or a patient already diagnosed with Alzheimer's disease to slow its progression and/or to reverse its effects (collectively referred to as treating Alzheimer's disease). Preferred compounds of the present invention for the treatment of Alzheimer's disease are the gamma-polyglutamate (γ-PGA) polymers preferably made by a biological process, such as, for example, the fermentation of Bacillus licheniformis. The molecular weight of the γ-PGA is from about 100,000 to about 7,000,000 Daltons and is usually from about 250,000 to about 2,000,000. Preferably, the molecular weight of the γ-PGA is about 1,000,000 Daltons. The γPGA can be fractionated to make a monodisperse γ-PGA. See FIG. 4. The γ-PGA can be fractionated employing standard techniques. By “monodisperse γ-PGA” is meant γ-PGA having a polydispersity of less than about 1.3, advantageously less than about 1.2 and preferably less than about 1.1. In a preferred embodiment a γ-PGA composition is made that is monodisperse having a P_{D} of about 1.0.

In another embodiment, the present invention is directed to the treatment of an apoptosis-mediated disease in a mammal, which comprises administering one or more compounds of Formulas I-IV, XII and/or the other compounds described herein in an amount that inhibits apoptosis. Additionally, the present neurodegenerative compounds described herein are employed to treat any disease state in a mammal that involves elevated nitric oxide synthase levels (NOS) and in particular NOS III. The present neurodegenerative compounds are administered in an amount that inhibits NOS activity.

In one embodiment of this invention, arabinogalactan is employed as the neuroprotective agent. Arabinogalactan, a water-soluble polysaccharide that can be isolated from species of the genus Larex. Arabinogalactan, is highly soluble and can be obtained at 95% purity from larch chips. In a preferred embodiment, refined arabinogalactan of above 95% or 99.9% purity is used as neuroprotective agent. The preparation of this material is disclosed in U.S. Pat. No. 5,116,969 and it is available from Larex, International, St. Paul, Minn. Arabinogalactan is highly stable, non-toxic, and non-immunogenic. As used herein, the term “arabinogalactan” includes naturally occurring or synthetic arabinogalactan, fragments of arabinogalactan, such as degradation products, and modified arabinogalactan or fragments thereof that have been modified using methods available in the art. As defined herein “refined arabinogalactan” refers to arabinogalactan with a purity greater than 95%. The molecular weight of arabinogalactan ranges from about 6,000 to 2,500,000 Daltons and for the refined arabinogalactan about 10,000-30,000 Daltons. Arabinogalactans derived from larch comprise predominantly a 1,3-β-D-galactan backbone with 1,6-β-D-galactobiose, 1,3-β-L-arabinofuranosyl-α-L-arabinofuranose, and α-L-arabinofuranose branch units and L-arabinose:D-galactose ratios of 1.6 to 1.7 (see Whistler R. L., Industrial Gums Whistler R. L. ed., p. 304, Academic Press, New York, 1993.). Other arabinogalactans are derived from acacia (gum arabic), and constitute complex, branched arabic acid derivatives (molecular weight of 250,000 to seven million Da) with a 1,3-β-D-galactose backbone with O-6 linked branches composed of two to five 1,3-β-D-galactosyl residues, α-L-arabinofuranose and 4-O-methyl-β-D-glucuronic acid units bearing additional L-rhamnose branches (see Whistler R. L., ibid., p. 311-318). Arabinogalactan formulations can be provided, for example, in the form of a sterile 50% solution diluted in a buffered isotonic salt solution and this solution can then either be used directly or mixed with DMSO. The preparation of endotoxin-free arabinogalactan has been described in U.S. Pat. No. 5,589,591.

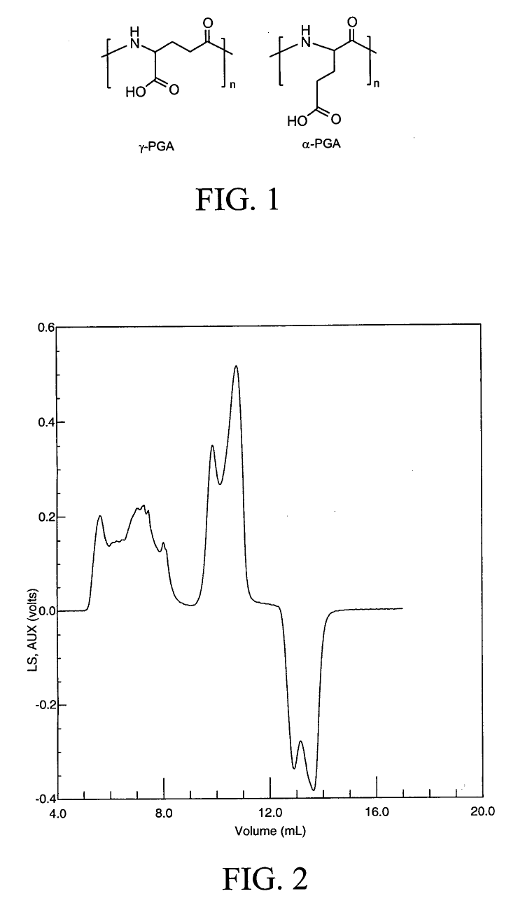

It is well established that the preparation of chemically derived poly(amino acids) faces serious challenges that have so far restricted the number of commercially available polymers, their purity, compositional homogeneity, reproducibility, polydispersity and molecular weight. Thermal polycondensation affords polymers with low degrees of polymerization (DPs of only up to 1,000) and broad polydispersity values (J. Vlasak, F. Rypacek, J. Drobnik and V. Saudek, J. Polym Sci. Polym. Symp., 66, 59, 1979.). Thermally derived poly(aspartic acid) is furthermore a copolymer of D- and L-isomers, containing both α- and β-peptide bonds in the main chain (H. Pivcova, V. Saudek, J. Drobnik and J. Vlasak, Biopolymers, 20, 1605, 1981.). Chemical polymerization of amino acid carboxyanhydrides on the other hand involves highly toxic chemicals, such as phosgene (W. D. Fuller, M. S. Velander and M. Goodman, Biopolymers, 15, 1869, 1976.), organic solvents and catalysts (H. Block, Poly[γ-benzyl-L-glutamate] and Other Glutamic Acid Containing Polymers, Gordon & Breach, New York, 1983; S. W. Shalaby (ed.), Biomedical Polymers, Designed to Degrade Systems, New York, Hanser, 1994; J. Kohn, R. Langer, Bioresorbable and Bioerodible Materials, in Biomaterials Science: An Introduction to Materials in Medicine, B. D. Ratner, et al. (eds), New York, Academic Press, pp 64-72, 1996.) and affords polymers with relatively low M_{w} values. Thus, the chemical polymerization of glutamate yields only the poly(α-glutamic acid) (α-PGA, see FIG. 1) with a very heterogeneous molecular weight distribution and average molecular weights below 50,000 Da. The heterogeneous nature of commercial α-PGA samples is illustrated in FIG. 2, which clearly reveals the presence of multiple α-PGA peaks with distinct molecular weight distributions. Furthermore, the currently available commercial α-PGA is extremely non-uniform in terms of its molecular weight characteristics, as demonstrated by SEC/MALLS analysis of three separate representative lots of α-PGA from a single supplier (Table 1). The results in Table 1 clearly show the poor reproducibility and broad molecular weight variability of commercial α-PGA. The hazardous nature and potential contamination of the chemically derived poly(amino acids) with trace levels of monomers, catalysts or solvents raises obvious additional concerns for their production and specially biomedical uses.

These inhomogeneities and structural variabilities of chemically derived poly(amino acids) are reflected in the study of Hefti et al. cited above. The authors attempted to examine a number of poly(amino acids) derived by thermal polycondensation of amino acid precursors and their ability to stimulate growth of dissociated fetal rat forebrain neurons (Hefti, F., et al., Brain Res., 541, 273-83, 1991). The authors studied either commercial homopolymers, e.g., α-polyglutamic acid with average molecular weights of up to 43,000 Da (from Sigma Chemical Co.) or copolymers (with Mw of 1,000-10,000 Da) containing multiple amino acid repeat units, e.g., aspartic acid, glutamic acid and tryptophan. The authors were unable to correlate the observed activities to polymer structures. They acknowledged substantial compositional heterogeneities of the polymers, such as the presence pigments and significant amounts of non-peptide linkages. They noted significant inconsistencies in the performance of these materials as a given poly(amino acid) could display variable potencies and be active or inactive, depending on the conditions of its preparations. Similarly, a poly(tryptophan) was inactive, whereas a copolymer of tryptophan and aspartic acid exhibited potency. The authors postulated that the presence of dicarboxylic acids was a prerequisite for activity. Hefti et al. also acknowledged close similarities in the activity of serum alone to those of their polymers.

In one embodiment of this invention, a γ-polyglutamate is employed as the neuroprotective agent. Various Bacillus species (e.g., B. licheniformis) elaborate the linear poly(γ-glutamic acid) (γ-PGA) (Y. Kanegae, Y. Sugiyama and I. Nakatsui, U.S. Pat. No. 5,268,279, 1993; I. L. Shih, Y. T. Van, Biosource Technology, 79, 207-225, 2001.), an unusual polypeptide with its glutamic acid residues linked through the γ-carboxyl function. γ-PGA assumes an α-helix conformation in solution, and, unlike the synthetic α-analogs, is a well-defined high molecular weight homopolymer. A further unique feature is that γ-PGA's composition can be varied from predominantly D-γ-PGA (>95%) to predominantly L-γ-PGA (>95%) by adjusting reaction conditions (e.g., by adjusting Mg concentrations). This allows for the additional control of biodegradability and other properties. γ-PGA is of particular interest for biomedical uses, due to its biocompatible, biodegradable (it degrades to glutamic acid), non-toxic, and non-immunogenic nature (R. H. De Kruyff, S. T. Ju, M. E. Dorf, Eur. J. Immunol., 17, 1115, 1987; Hutchinson, F. G.,

TABLE 1
Molecular weight (M_{w}) and polydispersity (P_{D})
of commercial α-PGA samples.*

| | Molecular weight | Polydispersity |
|---|---|---|
| Sample # | M_{w} (g/mol) | P_{D} (M_{w}/Mₙ) |
| 1 | 8,300 g/mol (±46%) | P_{D} = 1.395 ± 0.041 |
| 2 | 16,000 g/mol (±67%) | P_{D} = 1.270 ± 0.023 |
| 3 | 15,800 g/mol (±55%) | P_{D} = 1.538 ± 0.042 |

*determined by MALLS; M_{w} 15-50 kDa according to supplier information; data were obtained in 0.1 M phosphate buffer (KPOhd 4/K₂PO₄), flow rate 0.7 mL/min, Dawn DSP, 632.8 nm laser, dn/dc 0.15 mL/g.

B. J. A. Furr in A. H. Fawcett, ed., High Value Polymers, pp. 58-78, The Royal Society of Chemistry, Cambridge, England, 1990.).

Although γ-PGA is known for sometime, the γ-PGA employed for this invention was obtained by a specially developed, high yielding process that afforded γ-PGA with very high, previously unavailable purity (see Table 2 in Example 2 below).

Unlike the case of synthetic α-PGA, γ-PGA derived by fermentation can display molecular weights of up to 7,000,000 Da based on GPC/MALLS data (S. S. Mark, T. C. Crusberg, M. Yalpani, A. Dilorio. AIChE Annual Meeting, Los Angeles, October, 1997). The availability of high M_{w} γ-PGA constitutes a milestone in poly(amino acids) technology and is of substantial interest and potential benefit for biomedical applications. Despite its high M_{w}, PGA is also amenable to facile processing, as its solution viscosity can be substantially reduced by co-solutes, if required. Narrow molecular weight PGA fractions offer additional advantages.

Additionally, polyglutamate (PGA) can be used as a stool softener in a laxative composition. The term “laxative” is meant to include bulk laxatives (the various soluble and insoluble fibers), stool softeners (such as dioctyl sulfosuccinates) and stimulant laxatives (such as phenolphthalein and senna). A preferred polyglutamate is γ-PGA. The γ-PGA can have a molecular weight of from about 100,000 Daltons to about 7,000,000 Daltons and is usually from about 250,000 to about 2,000,000 Daltons. A preferred molecular weight of the γ-PGA is about 1,000,000 Daltons. For use as a stool softener the PGA is administered orally at a dose of 50 mg to about 1 gram or more. Usual doses are between 100 mg and 500 mg and a preferred dose is between 200 mg and 300 mg. The PGA can be administered alone or can be co-administered with bulk laxatives or stimulant laxatives. Preferably, the PGA is co-administered with one or more bulk laxatives either separately or in a composition that contains both the PGA and bulk laxative. The present PGA may also be combined with other stool softeners such as salts of dioctyl sulfosuccinate. Preferred salts include the sodium, calcium and potassium salts of dioctyl sulfosuccinate.

Bulk laxatives useful for co-administration with the present PGA include psyllium, calcium polycarbophil, brans (such as wheat bran, oat bran, rice bran, etc), malt soup extract, karaya, guar gum, methylcellulose and mixtures of the various fibers. Preferred bulk laxatives include psyllium, sodium methylcellulose, calcium polycarbophil, polycarbophil and mixtures thereof. A particularly preferred bulk laxative is ground psyllium husks such as commercially available METAMUCIL. The psyllium husks are preferably milled to a particle size where no more than 4% passes through a 100 mesh screen and 25% to 50% pass through a 200 mesh screen. Psyllium husk particle size ranges for use as a bulk laxative are described in U.S. Pat. No. 5,149,541 which is incorporated herein by reference.

The laxative compositions of the present invention may be any oral dosage form including, but not limited to, drink mixes, powders, capsules, tablets, and any kind of food or liquid incorporating the active ingredient(s) therein. When the present PGA is co-administered with a bulk laxative in a laxative composition containing both laxatives it is preferred to use a dosage form selected from the group consisting of powdered drink mix, a wafer, a cookie and a food bar. Optional ingredients to be added to these dosage formulations are well known to one of ordinary skill in the art. Such optional ingredients include sweeteners, flavoring agents, flow agents, starches, dextrin, maltodextrins, inert carriers, dispersants, emulsifiers, food colors and the like. Sweeteners include aspartame, sugar, saccharin, acesulfame K, sucralose and mixtures thereof. Laxative compositions and there preparation are described in U.S. Pat. No. 5,516,524 which is incorporated herein by reference.

A laxative composition of the present invention that contains γ-PGA having a molecular weight of about 1,000,000 Daltons and ground psyllium husks would contain the following active ingredients:

| | |
|---|---|
| Ground psyllium husks | 5 gm |
| γ-PGA | 250 mg |

The laxative composition is made with ground psyllium husks as described in U.S. Pat. No. 5,149,541 and then processed with maltodextrin and citric acid as described in U.S. Pat. Nos. 4,459,280 and 5,219,570 both of which are incorporated herein by reference. A preferred sweetener is aspartame or a mixture of aspartame with acesulfame-K, saccharin and/or sucralose.

In another embodiment a mixture of various bulk laxatives are substituted for the ground psyllium husks in the laxative composition described above in similar proportions with the γ-PGA. The bulk laxative mixture can contain two or more of the following bulk laxatives: ground psyllium husks, oat bran, rice bran, wheat bran, polycarbophil, calium polycarbophil, malt soup extrtact, karaya, guar gum and methylcellulose.

In another embodiment of this invention tripeptides are employed as the neuroprotective agent. Tripeptides are prepared employing procedures well known to one of ordinary skill in the art. The tripeptides encompassed under this invention include: arginine-glutamate-arginine, arginine-asparagine-arginine, lysine-glutamate-arginine, arginine-glutamate-lysine, ornithine-glutamate-arginine, arginine-glutamate-ornithine, citrulline-glutamate-arginine, arginine-glutamate-citrulline, N-acetyl-arginine-glutamate-arginine, arginine-glutamate-arginine-NH₂, arginine-glutamate-arginine-OCH₃, D-arginine-L-glutamate-arginine, L-arginine-D-glutamate-arginine and L-arginine-L-glutamate-arginine.

In yet another embodiment bridged aromatic compounds described by general formula IV [Y—(X)ₘ—R] are employed as the neuroprotective agent. The bridged aromatic compounds under this invention include: 4,4′-dichlorobenzydryl chloride, diphenylamine, 4-diphenylmethylene N-trifluoracetylpiperidine and Tamoxifen. Compositions of this invention are obtained from suitable precursors by established etherification, amination, reductive amination, or equivalent substitution reaction procedures known to those skilled in the art. Representative preparations of benzophenone-containing compositions are given below. Compositions containing the benzophenone moiety are prepared by conversion of the latter via borohydride reduction in alcohol solution to 1,2-bis(phenyl phenylmethoxy)ethane. The resulting benzhydrol (1 equivalent) is subsequently condensed with a suitable bifunctional alcohol or diol (e.g., ethylene glycol, 10 equivalents) in the presence of methanesulfonic acid catalyst to afford, after chromatography the corresponding ether or diether.

Representative examples of compounds of Formula IV encompassed by the present invention are given below:

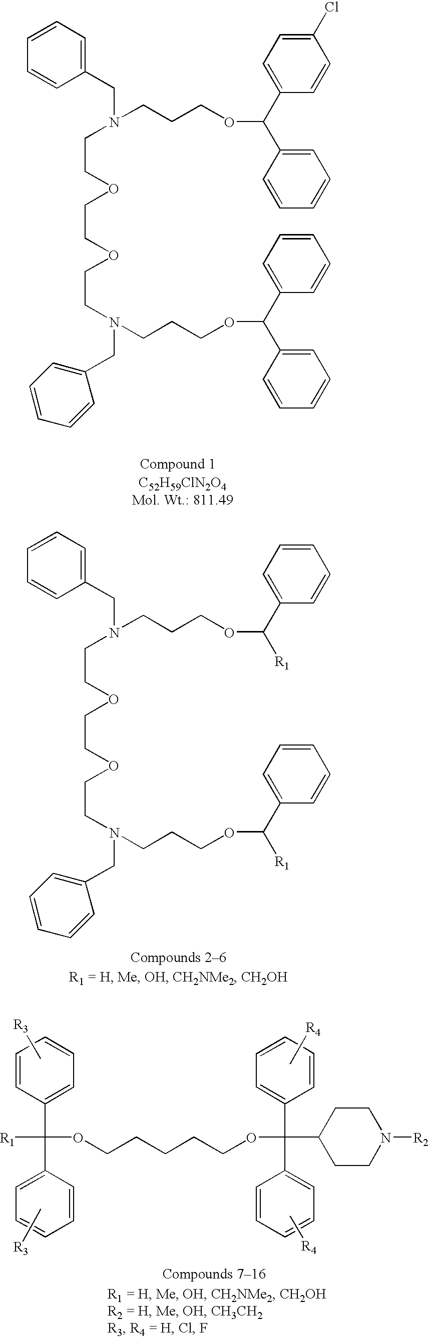

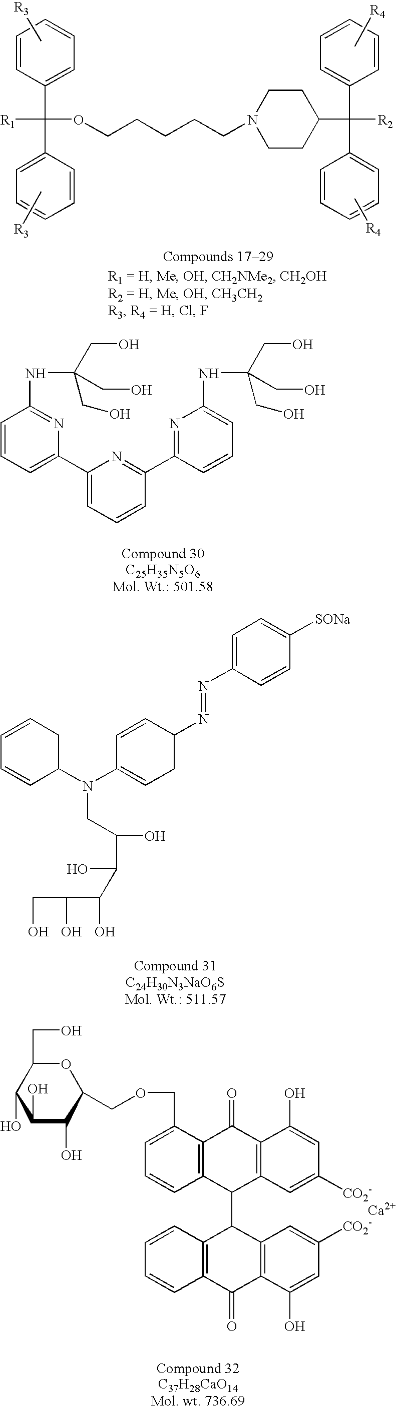

Further representative examples of compounds encompassed by Formula IV are given below:

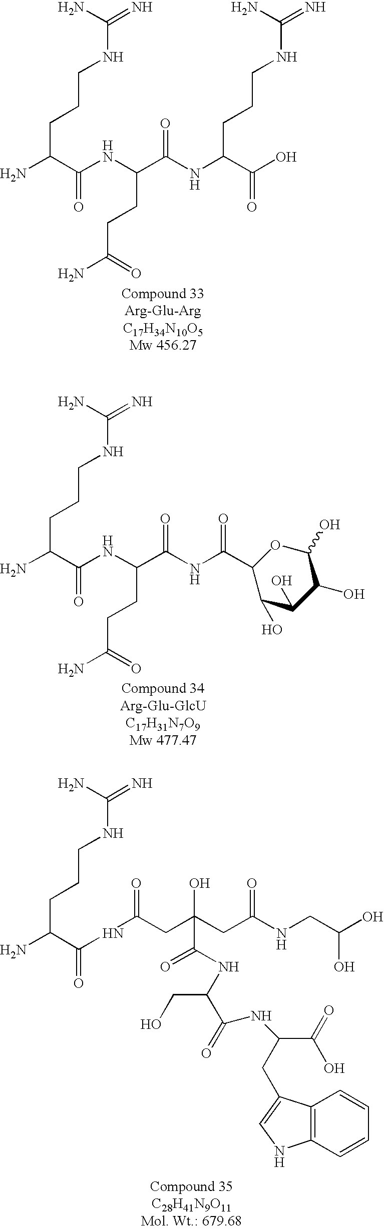

In yet another embodiment metal complexes and salts of biopolymers and synthetic polymers are employed as the neuroprotective agent. The polymer metal complexes under this invention include: polysaccharide iron complexes and γ-polyglutamate metal complexes. Additionally encompassed as part of the present invention are the following compounds that are also useful in the treatment of neurodegenerative diseases as disclosed herein:

p-Aminoclonidine; Aminopentamide; Amperozide; Atenolol; Atropine; Bepridil; Bietanautine 1,2,3,6-Tetrahydro-1,3-dimethyl-2,6-dioxo-7H-purine-7-acetic acid compound with 2-(diphenylmethoxy)-N,N-dimethylamine (2:1); 1-(2-(Bis-(4-fluorophenyl)methoxy)ethyl)-4-(3-phenylpropyl)-piperazine); 1-(2-(Bis-(4-fluorophenyl)methoxy)ethyl)-4-(3-phenyl-2-propenyl)-piperazine); Bromocriptine; Buaverine; Bulan; Buspirone HCl; Butyl-N-ethyl-2-(1-naphthyloxy); Clonidine; Ephedrine; Ethaneamine; Calmidazolium chloride; Carbamazepine; Cetinzne; Cetrizine; Chicago Sky Blue 6B; 1-(2-Chlorobenzoyl)piperazine-2,3-dicarboxylic acid, Na; 2-Chloro-11-(4-methylpiperazino)-dibenzy[b,f]oxepin]; 3α[(4-Chlorophenyl)phenylmethoxy]tropane; Cinnarizine; Ciprofloxacin; Clomipramine; Cyproheptadine; Deoprenilamine; Deofenine; Deprenyl; N-Desmethylclozapine; Diazepam; (2-(3,4-Dichlorophenyl)-N-methyl-[(1S)-1-phenyl-2-(1-pyrrolidoninyl)ethy](acetamide); (RS-[3-[1-[((3,4-Dichlorophenyl)acetyl]methylamino]-2-(1-pyrrolidoninyl)ethyl]phenoxy] acetic acid); N,N-Diethyl-2[4-phenylmethyl) phenoxy]ethanmine); 4,4′-Difluoro-3α-(diphenylmethoxy)tropane hydrochloride; 1,1-Dimethyl-4-diphenylacetoxypiperinium iodide (4-DAMP); Dimepheptanol; Diphenhydramine HCl; Diphenidol; Diphenolic acid; (1-(4,4-Diphenyl-3-butenyl)-3-piperidinecarboxylic acid); [1-(2-Diphenylmethoxyethyl)-4-(3-phenylpropyl)-piperazine); [1-(2-Diphenylmethoxyethyl)-4-(3-phenyl-2-propenyl)-piperazine); Diphenylpyraline; Dipipaone; Doxepin; Doxylamine; Droloxifene; Edrophenoium chloride; Emepronium bromide; Ergonamine; Ergotamine; Etastine; Etifelmin; Etodioxizine; Ethylbenzhydramine; Ethylbentropine; Felodidine; Fendiline; Feniprane; Fenoprofen; Fenpiverinium bromide; Fexofenadine; Fludpirilene; Flufenamic acid; Flunarizine; Fluoxetine; Flupentixol dihydrochloride; Fluphenazine-N-2-chloroethane; Flusipirilene; Galanthamine hydrobromide; Genistein; N-[3-(1-Hexahydroazapinyl)propyl]-a-cyclohexylbenzeneacetamide; Ifenprodil; Imipramine; Ipatropium bromide; Isomethadol; Isomethadone; Ketanserin tartrate; Ketotifen fumerate; Levomethadyl acetate; Lidoflazine; Lorazepam; Lupinifolin; Lupinifolinol; Meperidine; Methadone; Methadyl acetate; Methiothepin maleate; 2-[2-(4-(2-Methoyphenyl)piperazine-1-yl)ethyl]-4,4-dimethyl-1,3-(2H,4H)-isoquinolindione HCl; d-Methylphenidate; Mianserin HCl; Naftopidil dihydrochloride; Neostigmine; Noracymethadol; Normethadone; Norpipanone; Oxatomide; Oxazepam; Penfluridol; Pentacynium bis(methylsulfate); Pergolide; Phenadoxone; Phenoxybenzamine; Phentolamine; α-Phenyl-1-(2-phenethyl)-4-piperidinemethanol; Physostigmine; Pimozide; Piocarpine; Pirenzepine dihydrochloride; Propanolol; Pyridostigmine bromide; Rilmenidine; Rimcazole dihydrochloride; Ritanserin; Robinetin; 12-epi-Scalaradial; Scopilamine; Selegilline; Sennoside; Sertraline; Sulpiride; Tacrine; Tamoxifen; Tamoxifen, 4-hydroxy/3-hydroxy; N,N,N′,N′-Tetrakis-[(2-pyridylmethyl) ethylenediamine]; Thiothixene HCl; Trifluoroperazine; Trihexyphenidyl hydrochloride; 3-Tropanyl-3,5-dichlorobenzoate; Yohimbine; and Zimelidine dihydrochloride.

The neuroprotective agents of this invention can be used to treat or prevent neurodegenerative diseases. The neuroprotective agents are administered orally as tablet or capsule, intravascularly (for example, IM, IV or SQ) or intraperitoneally in physiological buffer or other physiologically acceptable carriers that are well known to one of ordinary skill in the art.

Preparation of New Neuroprotective Agents

### EXAMPLE A

### Tripeptides

Established synthetic methods for the preparation of polypeptides can be employed to obtain di-, tri-, tetra-, or higher polypeptides. A series of tripeptides, for example, was generated by the procedure given below. The preparation of a tripeptide set of compounds was accomplished, using a four-step protocol and the acid labile protected amino acid precursors, e.g., arginine, glutamate and asparagine:

Step 1. Coupling to solid support. Arginine, glutamate and asparagine (5 equivalents) were each reacted separately with the Wang resin (0.8 mmol/g) using HATU (2-(7-Aza-1H-benzotriazole-1-yl)-1,1,3,3-tetramethyluronium hexafluorophosphate, 5 eq) and DIEA (N,N-Diisopropylethylamine, 10 equivalents) as the coupling reagent and DMF/CH₂Cl₂ (1/1) as solvent. After 18 h shaking, the solid was washed with DMF (3 times), CH₂Cl₂ (4 times), MeOH (2 times) and ether (2 times), and dried under vacuum.

Step 2. Removal of Fmoc protecting group. The solid was treated with a solution of 20% piperidine in DMF (1 mL), shaken for 45 minutes then washed and dried under vacuum.

Step 3. Peptide coupling. The above two-step sequence was repeated twice with the product from Step 2 and the appropriate amino acids to give 27 tripeptides on the solid support.

Step 4. Cleavage from solid support. Each peptide was cleaved from the support by treatment with TFA/M₂O/triethylsilane (95:2.5:2.5) for 2 h. The solution was collected, the solid is washed with TFA (2 times) and these washes were added to the solution. This solution was cooled in an ice bath and treated with ether, and the solid was extracted with water (2 mL and 1 mL). The combined water layer was washed with ether (3 times) and freeze-dried to give the desired tripeptide. The products were identified by LC-Mass analysis.

### EXAMPLE 1

### Poly(γ-Glutamic Acid)

Bacillus licheniformis ATCC 9945 from 1 mL freezer stocks was inoculated into starter medium (50 mL, 10 g/L peptone, 2 g/L yeast extract, 0.02 g/L MgSO₄.7H₂O.) and cultured to an absorbance (at 600 nm) of 1.0-1.5, ˜8 h, to inoculate starter cultures for fermentations. Cells grown in production overnight (0.5 mL) were mixed with a 0.5 mL of a 50% glycerol solution and the cryovials were stored at −80° C. A single B. licheniformis ATCC 9945 cryovial was added to 250 mL (seed culture) production medium: 80 g/L glycerol, 50 g/L citric acid H₂O, 50 g/L L-glutamic acid, 8.63 g/L NH₄SO₄, 0.5 g/L K₂HPO₄, 0.5 g/L MgSO₄7H₂O, 0.08 g/L MnSO₄H₂O, and 0.15 g/L CaCl₂2H₂O, pH adjusted to 7.0 with NaOH. Seed culture was added to a New Brunswick Bioflo III containing production medium (5 L) at a ratio of 4%. The fermentation conditions were: dissolved oxygen (D.O.) 5%, temperature 37° C., agitation 300-500 rpm (under active D.O. control), no pH control, antifoam as required (Antifoam 204). The fermentation progressed at 37° C. for 3 days and was harvested by first acidifying (HCl) the broth (pH 2.0) to facilitate the broth flow through the tubing. The reduced pH broth was then centrifuged at 16,000×g for 20 minutes.

To obtain high M_{w} PGA, the supernatant was neutralized (NaOH), and then dialyzed twice against 2 mM NaEDTA (12,000 MWCO dialysis tubing) and three times against deionized water, yielding γ-PGA with M_{w} 1·10⁶ Da.

### EXAMPLE 2

### Fractionation of Poly(γ-Glutamic Acid)

To fractionate high M_{w} γ-PGA of Example 1, the fermentation supernatant (pH 2.0) was fractionated with a tangential flow microfiltration apparatus. Filtrate samples, analyzed by GPC/MALLS indicated a M_{w} reduction to 300,000 Da. Alternatively, a Microfluidizer Cell Disrupter model M-110-T (Microfluidics, Inc.) was used at various pressures (5,000, 10,000 or 15,000 PSI) and reduced the M_{w} to 300, 180 and 380 kDa, respectively, with no P_{D} change (as determined by MALLS).

### EXAMPLE 3

### Fractionation of Poly(γ-Glutamic Acid)

The fractionated γ-PGA of Example 2 was further processed by ultrafiltration, using a 100,000 MWCO regenerated cellulose filter. As filtration progressed, a further M_{w} reduction to 15,000 Da was achieved.

Poly(γ-glutamic acid) Purity. The material prepared in Example 1 was examined for its purity. The results are shown in Table 2, and compared with a prior art material. The results show clearly that γ-PGA in Example 1 is of superior purity.

TABLE 2
Purity Profile Comparison of γ-PGA (from Bacillus licheniformis)*

| | | Concentration | |
|---|---|---|---|
| | | (per mg of purified γ-PGA) | |
| Material | Source | Example 1 | McLean et al.** |
| Protein | Cell component | 0.81 μg | 14.97 μg |
| | proteins | | |
| Organic | Membrane | 9.5 nmol | 100 nmol |
| Phosphate | phospholipids, nucleic | | |
| | acids, or teichoic acids | | |
| Uronic Acids | Teichuronic acids | 0.63 μg | 8.37 μg |
| Heavy Metals: | | | |
| Copper | | 127 pmol | 500 pmol |
| Zinc | | 62 pmol | Not Determined |
| Cadmium | | <18 pmol | Not Determined |

*Results shown are for a γ-PGA capsule preparation obtained by McLean et at and for a preparation of native γ-PGA obtained as described in Example 1.
**McLean, R. J., Beauchemin, D., Clapham, L., Beveridge, T. J., J. Appl. Environ. Microbiol., 56, 3671-3677, 1990.

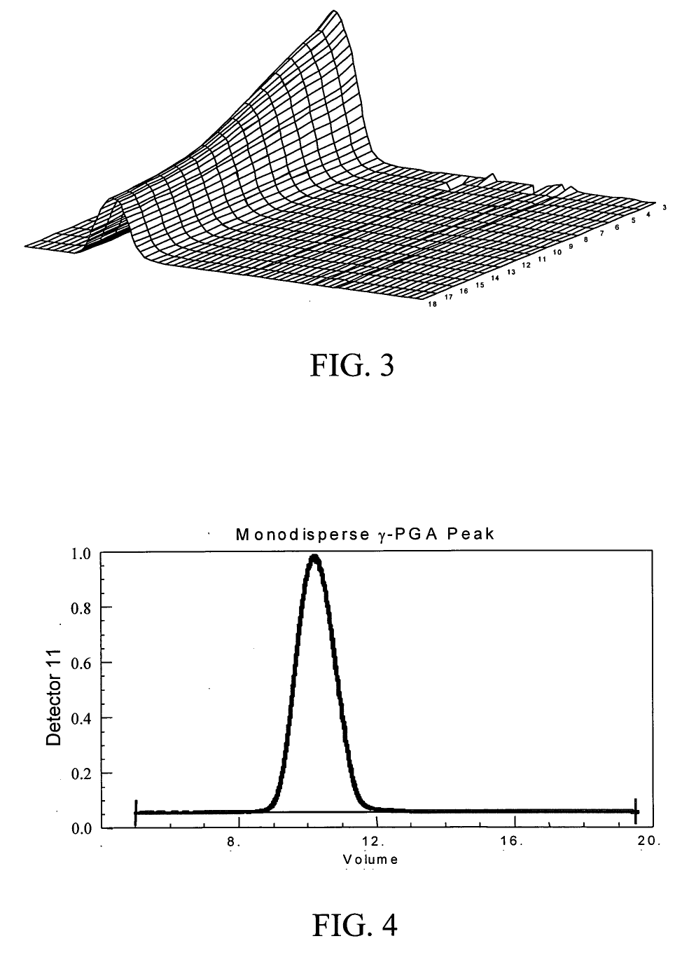

Poly(γ-glutamic acid) Molecular Weight Profile. The γ-PGA materials prepared in Examples 1 and 2 were examined for their molecular weight characteristics, using SEC/MALLS. The chromatograms obtained are shown in FIGS. 3 and 4, respectively, and clearly reveal the high uniformity and narrow molecular weight distribution (P_{D} values of 1.4 and 1.0, respectively) of the native and fractionated materials. The fractionated γ-PGA was monodisperse, a feature that has previously been inaccessible for this biopolymer. A comparison with the properties of α-PGA demonstrate how advantaged and ideally suited the novel γ-PGA materials are for biomedical applications. Note that at the lower angles (lower detector number), intensity of response to the polymer is increased.

Toxicity Studies of Native γ-PGA. Toxicity studies were performed, using purified γ-PGA (1×10⁶ Da material obtained in Example 1) in rat muscle (L6) and rat liver (H35) cell lines at concentrations ranging from 5 mg/L-0.5 g/L in Dulbecco's Modified Eagle's Medium (DMEM). Once cell lines were established, media exchanges were performed with DMEM containing γ-PGA at the appropriate concentration. The L6 cell lines all differentiated normally and γ-PGA did not interfere with differentiation into the elongated muscle form. Results from the H35 cell line study indicated again, no significant adverse effect on cell growth.

The in vitro cytotoxicity of γ-PGA was also determined, using the Brine Shrimp Lethality Assay at γ-PGA concentrations of 10, 100, and 1,000 μg/mL. No cytotoxicity was observed, using unmodified Taxol as comparative standard.

### EXAMPLE 4

### Benzohydrol

Benzophenone was converted via borohydride reduction in alcohol solution to 1,2-bis(phenyl phenylmethoxy)ethane. The resulting benzhydrol (1 equivalent) was subsequently condensed with a suitable bifunctional alcohol or diol (e.g., ethylene glycol, 10 equivalents) in the presence of methanesulfonic acid catalyst to afford, after chromatography the corresponding ether or diether.

### EXAMPLE 5

### 1,2-Bis[di(phenyl)methoxy]ethane

The benzhydrol (1 equivalent) and triethylamine (1.2 equivalent) in ether was treated in an ice bath with methanesulfonyl chloride to afford the benzhydrol methanesulfonate. The latter was then treated with ethylene glycol (or alternative nucleophile, e.g., amine) and ethyldiisopropylamine to afford 1,2-bis[di(phenyl)methoxy]ethane (or corresponding amine).

### EXAMPLE 6

### 1-Di(phenylmethoxy)-2-(diphenylmethoxy)-ethane

The benzhydrol (1 equivalent) in dichloromethane was cooled in an ice bath, and concentrated hydrochloric acid (10 equivalent) was added slowly and stirred in the ice bath for 1 h and then overnight at room temperature. The dichloromethane solution was washed twice with brine, dried over sodium sulfate and evaporated under reduced pressure to afford benzhydryl chloride as an oil. This intermediate was then condensed with suitable synthons to afford bisether or ether derivatives. Thus, 2-diphenylmethoxyethanol (1.5 equivalent), benzhydryl chloride (1 equivalent) and ethyldiisopropylamine (1 equivalent) were mixed and the reaction mixture was heated. Upon completion of the reaction, the mixture was cooled, diluted with dichloromethane, and washed with water (3 times). The organic layer was filtered and evaporated to give a crude product that was purified by chromatography, affording 1-di(phenylmethoxy)-2-(diphenylmethoxy)-ethane.

### EXAMPLE 7

### 2-Diphenylmethoxyethyl p-toluenesulfonate

Benzhydryl chloride (2 equivalents) in acetonitrile was added dropwise to a mixture of piperazine (1 equivalent) and potassium carbonate (0.3 equivalent) in acetonitrile and stirred at reflux for 4 h. The reaction mixture was filtered, the collected solid washed with acetonitrile and the combined filtrates were concentrated and then mixed with 1 M aqueous sodium hydroxide. The mixture was extracted with dichloromethane, the latter phase was then extracted with 1 M aqueous sodium hydroxide, filtered, concentrated in vacuo, dried and purified by chromatography to afford an oil. The oil was mixed with a small volume of hexanes to produce a crystalline solid.

An alternative intermediate, 2-diphenylmethoxyethyl p-toluenesulfonate was obtained from the corresponding alcohol and tosyl chloride. This intermediate was condensed with suitable amines.

### EXAMPLE 8

### [(1,2-Dianilino)-1,2-di-(1-Amino-1-deoxy-D-sorbitol)]-ethane

One equivalent of 1,2-dianilinoethane was condensed with two equivalents of glucose in a dichloromethane for 12 h to afford after recrystallization [(1,2-dianilino)-1,2-di-(1-amino-1-deoxy-D-sorbitol)]-ethane, C₂₆H₄₀N₂O₁₀, Fw 540.60.

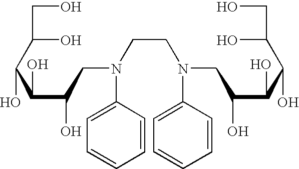

### EXAMPLE 9

### 1,2-Dideoxy-[1-(benzophenone hydrazide)-2-(phenylmethane)]-D-glucit-1-yl

Benzophenone hydrazone (1.1 equivalent) was condensed with one equivalent of glucosamine in a dichloromethane in the presence of sodium cyanoborohydride (11 equivalents) for 12 h. The reaction product was further treated with benzaldehyde (1.5 equivalent), affording after chromatography, 1,2-dideoxy-[1-(benzophenone hydrazide)-2-(phenylmethane)]-D-glucit-1-yl, C₂₆H₃₁N₃O₄, Fw 449.54.

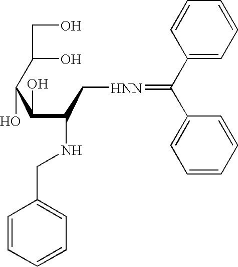

Using methods known to those skilled in the art, the compounds identified in Examples 10-16 (Table 3) were prepared, which further illustrate the scope of the invention.

TABLE 3

| Example # | Compound | Formula | Fw |
|---|---|---|---|
| 10 | 2-[(4-Phenylmethyl)phenoxy)-1- (trishydroxymethyl)aminomethane}ethane 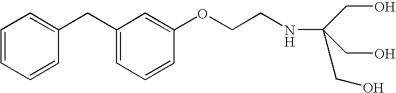 | C₁₉H₂₅NO₄ | 331.41 |
| 11 | [(1-(4,4-Diphenyl-3-butenyl)-3- piperidinecarboxylic acid)-2-(trishydroxymethyl)- aminomethane]ethane 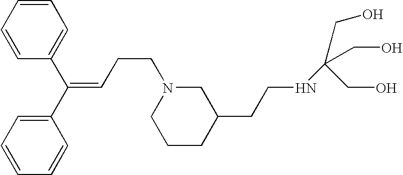 | C₂₇H₃₈N₂O₃ | 438.60 |
| 12 | 1-[(1,1,1-Triphenylmethyl)-2-hydroxy-5 carbonyl-6-(trishydroxymethyl)- aminomethane]hexane 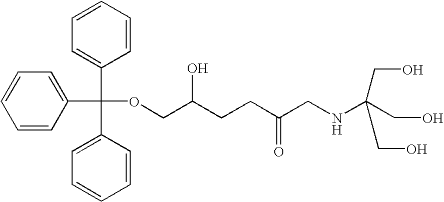 | C₂₉H₃₅NO₆ | 493.59 |
| 13 | 1-[2-(1,1,1-Triphenylmethylamine)-3-carbonyl-4- (trishydroxymethyl)-aminomethane]butanol 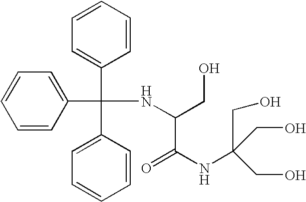 | C₂₆H₃₀N₂O₅ | 450.53 |
| 14 | (1-Methoxy-4-benzenesulfonylamine)-2-(N-ethyl- 2-pyrrolidinylmethyl)-carboxamide 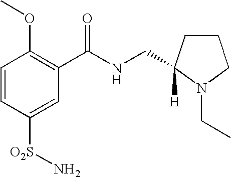 | C₁₅H₂₃N₃O₄S | 341.43 |
| 15 | 1,9-Bis[(1,9-diamino-4,7-dioxa)(—N,N′- phenylmethyl-[(4- diphenylmethoxy)butane]decane 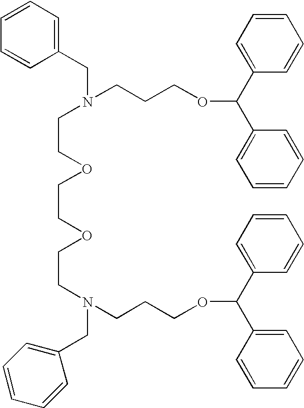 | C₅₂H₆₀N₂O₄ | 777.04 |
| 16 | [(1-Hydroxy-1,1-diphenylmethoxy)-(1-{4-N- methylpiperidine}-1,1-diphenylmethoxy)]heptane 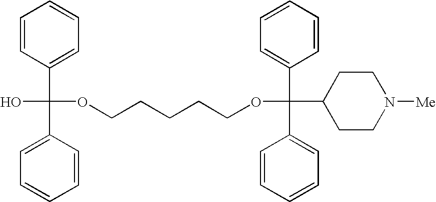 | C₃₇H₄₃NO₃ | 549.74 |

### EXAMPLE 17

### Biological Screening Tests for Neuroprotective Agents

For the study of CNS neuron apoptosis an array of in vitro and in vivo high through-put screening tools were used with primary neuronal cell cultures to assess the therapeutic potential of the test compounds. These tests permit the quantitation of NOS III protein levels, over-expression of NOS III with recombinant adenovirus, determination by several methods of NOS III-mediated apoptosis in neuronal cells, measurement of cell viability and sensitivity to oxidative stress, and assessment of pro-apoptosis gene activation and survival gene inhibition with the Microtiter Immunocytochemical ELISA (MICE) assay.

High through-put screening using neuronal cells. The screening tests were conducted using PNET2 neuronal cells of human CNS origin. These are advantaged because they exhibit many properties of human cortical neurons including: 1) growth factor-induced neurite outgrowth and neuronal gene expression; 2) intact intracellular growth-factor signaling; 3) activation of the same apoptosis genes and signaling pathways observed in vivo; 4) relative ease of gene transfer by transfection or infection; and 5) low endogenous levels of NOS III. Two major approaches were utilized: 1) PNET2 cells were infected with different multiplicities of infection (MOI) (0-200) of AdvNOS III or Adv-GFP; and 2) PNET2 cells were infected with 0, 10 or 20 MOI of Adv-NOS III or Adv-GFP, and treated with low, non-toxic concentrations of H₂O₂ (8 μM), desferoxamine (0.1-2 mM), or DDC (200 μM). H₂O₂ provides a source of superoxide and free radicals. Desferoxamine was added as it provides an in vitro model of hypoxic/ischemic injury, and treatment of PNET2 cells with 1-10 mM desferoxamine induces apoptosis. DDC inhibits production of free radical scavenger agents. Viability was measured by the MTT assay. In addition to assaying viability, the degree to which the synthetic compounds inhibit NOS III-induced in situ apoptosis, DNA damage, activation of pro-apoptosis genes, and inhibition of cell survival genes was assessed.

Neuronal culture conditions. PNET2 cells were maintained in Dulbecco's modified Eagle's medium (DMDM) supplemented with 0.9% glucose, 8 mM glutamine, 100 μM non-essential amino acids, and 10% fetal bovine serum. Subconfluent cultures seeded into 96-well plates (2×10⁴ cells/well) were infected with recombinant Adenovirus vectors (Adv) that express a full-length cDNA encoding NOS III or green fluorescent protein (GFP) under control of a CMV promoter. The cells were studied at 0, 24, 48, or 72 hours after infection. GFP and NOS III expression was verified by the microtiter immunocytochemical ELISA (see below). In addition, GFP expression was visualized by fluorescence microscopy. In parallel experiments, PNET2 cells were infected with 20 MOI of recombinant adenovirus, and 24 hours later when gene expression was detectable, in >80% of the cells, the cultures were exposed to low levels of H₂O₂ (8 μM), desferoxamine (0.1-2 mM), or DDC (200 μM).

Cell viability assays. Cell viability was measured by the 3-[4,5-dimethylthiazol-2-yl]-2,5-diphenyltetrazolium bromide (MTT) assay, using cells seeded into 96-well plates. The MTT assay is based on conversion of MTT to formazin by a mitochondrial enzyme. During the last 4 hours of maintenance or treatment, the cells were incubated with 0.5 μg/ml of MTT solution prepared in phenol red-free DMEM. The plates were rinsed in PBS and the MTT formazin precipitate was eluted into 50 μl/well of 0.04 N HCl in methanol. The absorbance was read at 540 nm, and background readings at 670 nm were subtracted. MTT absorbance values were linear with respect to cell density between 10⁴ and 5×10⁵ cells/well. Since the MTT assay measures both metabolic function (mitochondrial-based) and cell viability, a second method, the crystal violet assay, was used to assess cell density. For the crystal violet assay, the cells were rinsed in phosphate buffered saline (PBS) and stained for 10 minutes at room temperature with crystal violet solution (0.05% crystal violet, 2% formalin, 10% methanol). After extensive rinsing in tap water baths, the plates were dried and the dye eluted with 200 μl/well of PBS containing 1% SDS. The absorbance was measured at 595 nm using an automated ELISA reader.

Apoptosis assays. Several methods to detect apoptosis were used as there are now markers that detect very early, intermediate, and late changes preceding nuclear DNA fragmentation. As an early marker of apoptosis, immunoreactivity for Annexin V screened using fluorescein-conjugated Annexin V. After incubation with FITC-conjugated Annexin V, the cells were fixed and stained with DAPI. Using an automated fluorescence reader, the relative abundance of Annexin V-positive cells (DAPI-positive cells as denominator) was assessed. For detecting nicked DNA, the cells were stained with antibodies to single-stranded DNA (Apostain) by the MICE assay. To detect later stages of apoptosis, the cells were labeled with antibodies to peroxynitrite since peroxynitrite is a free radical generated by reaction of NO and superoxide. Peroxynitrite immunoreactivity was assessed by the MICE assay (see below).

Assessment of pro-apoptosis gene activation and survival gene inhibition with the Microtiter Immunocytochemical ELISA (MICE) Assay. The MICE assay is a rapid method for objectively quantifying levels of immunoreactivity in cultured cells without the need for protein extraction, gel electrophoresis, or cell counting. The MICE assay differs from the cellular ELISA in that it incorporates a correction for cell density into the procedure, thereby permitting comparisons of protein expression following different treatments, even if the effect of the treatment includes cell death or proliferation. In addition, the MICE assay measures levels of protein expression, whereas cellular ELISAs are designed to detect surface immunoreactivity. Detailed methods describing this assay were described recently (de la Monte S. M., Ganju N, Wands J. R., Biotechniques, 26, 1073-1075, 1999). At the conclusion of the experiments, the cells are fixed in Histochoice solution, then permeabilized with 0.05% saponin in Tris-buffered saline, pH 7.4 (TBS). Endogenous peroxidase activity was quenched with 0.03% H₂O₂. Non-specific binding sites were blocked with Superblock-TBS (Pierce). The cells were incubated overnight at 4° C. with primary antibody diluted to 0.5 μg/ml in TBS containing 0.05% Tween 20 and 0.5% bovine serum albumin (TBST-BSA). Antibody binding was detected with horseradish peroxidase conjugated anti-mouse IgG and TMB soluble peroxidase substrate (100 μl per well). Color development was stopped prior to saturation by adding 100 μl 1 M H₂SO₄ per well. The absorbance was measured at 450 nm using an automated microplate reader.

To accurately assess the levels of protein expression, it is necessary to correct for differences in cell density among the wells. Fixed cells were uniformly stained with Coomassie blue dye (0.05% Coomassie blue in 40% methanol/10% acetic acid) and the absorbance values (650 nm) increased linearly with cell density between 1×10⁴ and 5×10⁵ cells per well (R²=0.997). To assess cell number after measuring immunoreactivity, the cells were rinsed with TBS, and stained for 10 minutes with 50 μl per well of Coomassie blue dye. The plates were washed extensively in warm tap water and then air-dried. Proteins complexed with dye were solubilized in 200 μl of 1% SDS in TBS. The absorbance at 650 nm was measured, using an automated microplate reader. The MICE index was calculated from the ratio of the absorbances for immunoreactivity and Coomassie blue labeling (cell density). The means and standard deviations obtained for 8 replicate culture wells were used in the data analysis. Using the MICE assay, levels of NOS III, GFP, p53 (pro-apoptosis), Bcl-2 (survival), and Bax (pro-apoptosis) were measured. Positive and negative controls for antibody binding were included in all assays.

High through-put screening with primary neuronal cell cultures. Primary neuronal cells were cultured in 96-well plates coated with collagen type IV or laminin. The primary neuronal cultures were infected with recombinant adenovirus vectors and gene expression measured, using the MICE assay. Finally, the rat primary neuronal cell cultures exhibit p53-mediated apoptosis after exposure to H₂O₂, desferoxamine, or DDC.

Cerebral cortex tissue harvested from 16-18 day old rat fetuses (Sprague-Dawley) was incubated for 10 minutes in calcium-free and magnesium-free Hanks balanced salt solution (CMF-HBSS), followed by digestion in 0.25% trypsin/CMF-HBSS for 30 minutes at 37° C. After washing in Eagle's basal medium containing 1% ovalbumin, the cells were dissociated by trituration. The cells were suspended in Eagle's basal medium plus N1 supplement (defined medium with 0.83 μM insulin, 0.062 μM transferrin, 10 mM putrescine, 0.02 μM progesterone, and 0.03 μM selenite) and seeded at a density of 5×10⁴ cells/well of 96-well plates. The wells were pre-coated with polyornithine (0.1 mg/ml poly-(L-ornithine) in 15 mM borate buffer, pH 8.4 for 2 h at room temperature), and after rinsing with DMEM, adding a coating of laminin (1 μg/ml; overnight) to promote cell adhesion and neurite outgrowth. Because of the developmental stage, the majority of cells were neuronal.

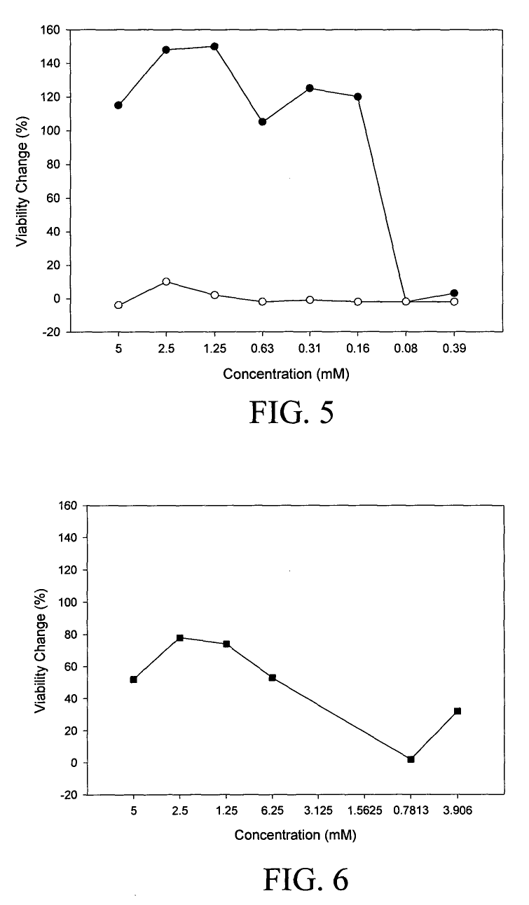

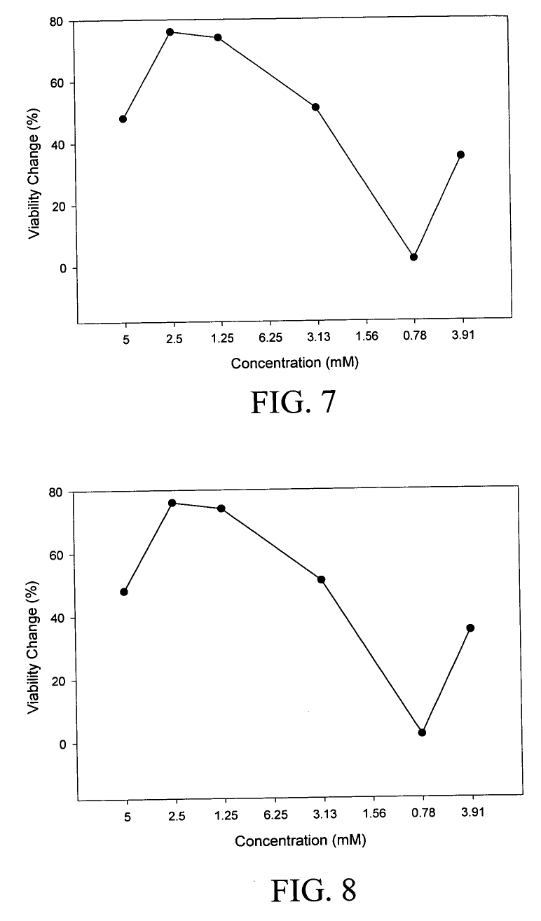

Test Results of Neuroprotective Agents. The neuroprotective activities of the compounds of this invention are as follows: When the changes in neuronal viability were measured as a function of γ-PGA concentration it was observed that γ-PGA delivered very significant rescue efficacy to neuronal cultures that had previously been subjected to oxidative stress (40 mM H₂O₂). In these experiments, primary cortical neuron cultures were pre-treated with various dilutions of γ-PGA one day prior to being subjected to the oxidative injury. The cell viability was measured using the crystal violet assay one day after the oxidative injury. Oxidant-exposed cultures exhibited a 40% loss of viability and mitochondrial function. The standard deviations calculated for 8 replicate cultures were <10% in all instances. γ-PGA pre-treatment consistently resulted in complete neuronal rescue and viability increases over a broad concentration range. The neuronal viability increased by up to 180% (expressed as percentage change relative to cultures treated only with the oxidant) for millimolar concentrations (1.2-2.5 mmol) for high molecular weight γ-PGA (1 million Da) and up to 15% for low M_{w} γ-PGA (300,000 Da) at similar concentrations (1.2-2.5 mmol). See FIGS. 5 and 7. In FIG. 5 “•” represents data from γ-PGA and “o” represents data from the control. Points falling below the abscissa reflect further cell loss, while points above the abscissa indicate improved viability.

In similar experiments, arabinogalactan pre-treatment resulted in complete neuronal rescue. The neuronal viability increased by up to 80% for millimolar concentrations (1.2-2.5 mmol) of arabinogalactan. FIGS. 6 and 8 show changes in neuronal viability as a function of arabinogalactan concentration: rescue efficacy of arabinogalactan delivered to neuronal cultures subjected to oxidative stress (40 mM H₂O₂). Viability is expressed as percentage change relative to cultures treated only with the oxidant. Primary cortical neuron cultures were pre-treated with various dilutions of arabinogalactan 1 day prior to being subjected to oxidative injury. Cell viability was measured using the crystal violet assay 1 day after the oxidative injury. Oxidant-exposed cultures exhibited a 40% loss of viability and mitochondrial function. The graphs illustrate the percentage change in viability relative to cultures treated with the oxidant only. The percentage changes in viability relative to oxidant-only treated cultures are depicted by the line graphs. Points falling below the abscissa reflect further cell loss, while points above the abscissa indicate improved viability. Arabinogalactan pretreatment resulted in complete neuronal rescue and produced a significant increase in viability over a broad concentration. Standard deviations calculated for 8 replicate cultures were <10%.

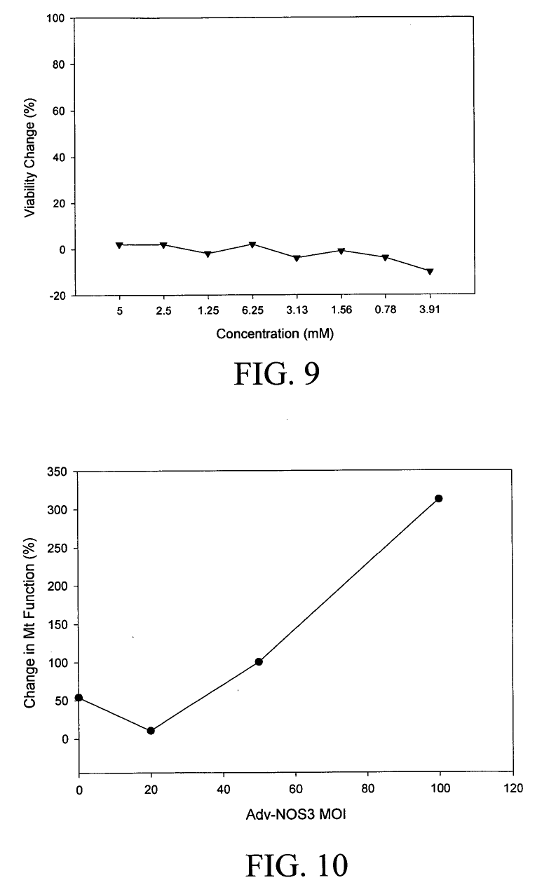

Diphenylamine pretreatment resulted in less pronounced neuronal rescue at similar concentrations. See FIG. 9 which shows changes in neuronal viability as a function of diphenylamine concentration in tests similar to those described above. Diphenylamine pretreatment produced a modest increase in viability.

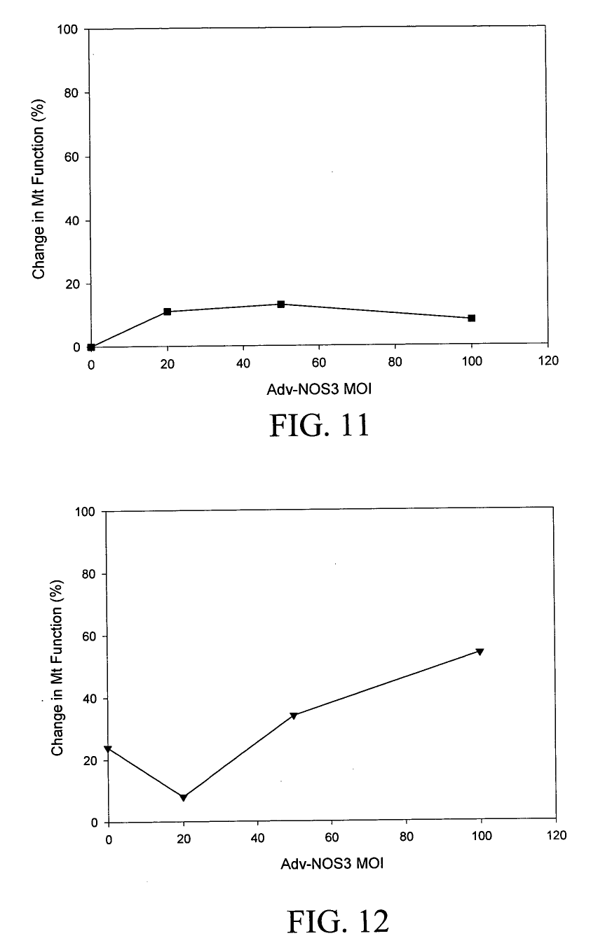

Similar trends were observed when the change in mitochondrial function was assessed as a function of increasing multiplicity of adenovirus infections (MOIs). See FIGS. 10-12. In these experiments, primary cortical neuron cultures were infected with recombinant adenovirus (Adv) expressing NOS III or green fluorescent protein (GFP, as negative control). NOS III over-expression at different MOIs resulted in 20-40% loss of mitochondrial function and cell viability, whereas identical Adv-GFP concentrations did not affect culture viability or mitochondrial function. Since Adv expression occurs approximately one day after infection, the cultures were treated with neuroprotective agent one day after Adv inoculation. Viability and mitochondrial (Mt) function were measured one day later. The Mt function was assessed by the MTT assay (described above) and changes in Mt function were calculated relative to control cultures not exposed to neuroprotective candidates. The graphs illustrate the viability change relative to cultures infected only with Adv-NOS III. Points falling below the abscissa reflected further cell loss, while points above the abscissa indicated improved viability. Standard deviations calculated for 8 replicate cultures were below 10% in all instances. The rescue efficacy was assessed for neuroprotective candidate treatment for neuronal cultures subjected to oxidative stress via nitric oxide synthase 3 (NOS III) over-expression. FIG. 10 shows that γ-PGA treatment again resulted in increased Mt function (up to 320%) in un-infected cultures. With increasing MOIs (0-100), γ-PGA exerted neuroprotective effects. FIG. 12 shows that arabinogalactan treatment resulted in increased Mt function (up to 60%) in un-infected cultures. With increasing MOIs (0-100), arabinogalactan exerted neuroprotective effects. FIG. 11 shows that dichlorobenzhydrol phenoxyamine provided less pronounced neuroprotection against NOS III over-expression under the experimental conditions.

Uses of Novel Neuroprotective Agents. This invention relates to compounds represented by the general formulas (I-IV, XII and other compounds described herein) that have a nitric oxide synthase (NOS) inhibiting effect or neuroprotective effect and thereby prove effective in ameliorating, preventing or reversing degenerative or neurological diseases and disorders resulting from excessive nitric oxide (NO) or its metabolites, including cerebrovascular diseases, such as cerebral hemorrhage, subarachnoid hemorrhage, cerebral infarction (atherothrombotic infarction, lacunar infarction and cardiogenic embolism), transient ischemic attack and cerebral edema, traumatic brain injury, spinal injury, pain, such as headache (migraine, tension headache, cluster headache, chronic paroxysmal headache), neurodegenerative diseases, such as Alzheimer's disease, Parkinson's disease, Pick's disease, Creutzfield-Jacob disease, Amyotrophic Lateral Sclerosis (ALS), Multi-infarct dementia, Luetic brain disease, epilepsy, Huntington's disease, diffuse Lewy Body disease, Down's Syndrome, Korsakoff's disease, subdural hematomas, occult hydrocephalus, Gilles de la Tourette syndrome and tardive dyskinesia; disorders of the central nervous system, such as ischemia and stroke, mood and emotional disorders, such as depression, pernicious anemia, Scrapie, panic, anxiety and psychosis; substance abuse, including withdrawal syndromes, substitution therapy, morphine tolerance or dependence; thyroid disease, HIV infection, neuroendocrine disorders and dysregulation of food intake, including bulimia and anorexia; seizure, disorders of nociception and pain control; autonomic disorders, including dysfunction of gastrointestinal motility and function, such as inflammatory bowel disease, irritable bowel syndrome, diarrhea, constipation, gastric acid secretion and ulcers; pheochromocytoma; cardiovascular dysfunction, including hypertension and cardiac arrhythmias, co-medication in surgical procedures; septic shock, chronic rheumatoid arthritis, osteoarthritis, viral or non-viral infections and diabetes mellitus. The invention further relates to therapeutics that contain said neuroprotective agents, derivatives or pharmaceutically acceptable salts as active ingredients.

Compounds of the general formulas I-IV and XII and the other compounds described herein and the pharmaceutical compositions derived from these compounds can also be used for the post-acute therapeutic treatment of a variety of neurological conditions in which various cell types of the nervous system are degenerated and/or have been damaged as a result of injuries or exposures. In particular, the present neuroprotective compounds can be used for the treatment of resulting conditions, in which damage to cells of the nervous system has occurred due to surgical interventions, infections, exposure to toxic agents, tumors, nutritional deficits or metabolic disorders. In addition, the present neuroprotective compounds can be used for the treatment of the sequelae of injuries, dystrophy or degeneration of the neural retina (retinopathies) and peripheral neuropathies, such as diabetic neuropathy and/or the peripheral neuropathies induced by toxins. The present neuroprotective compounds can also be used in combination with surgical implantations of tissues and/or prostheses for the treatment of Alzheimer's disease or other neurological disorders and/or malfunctions in which implantation is indicated.

The prophylactic and therapeutic agents of the present invention can be optionally provided in any dosage form known in the art that can be manufactured by a known pharmaceutical technology that comprises, for example, mixing or dissolving the active compound with a pharmaceutically acceptable carrier or vehicle. Of such dosage forms, the oral dosage forms for use in humans include powders, granules, tablets, capsules, syrups, and other liquid preparations. Powders, granules, tablets and the like can be manufactured using optional pharmaceutically suitable carriers that are suitable for solid preparations, such as excipients (e.g., starch, glucose, fructose, sucrose, lactose, etc.), lubricants (e.g., magnesium stearate, calcium stearate, etc.), disintegrators (e.g., starch, crystalline cellulose, etc.), binders (e.g., starch, gum arabic, etc.), and so forth. Such solid preparation may be optionally coated with a coating agent (e.g., gelatin, sucrose, etc.) or an enteric coating (e.g., hydroxypropyl methylcellulose phthalate, methacrylic copolymers, shellac, etc.), so that the active compound may be released specifically in the intestines. For the manufacture of syrups and other liquids, various additives such as stabilizers (e.g., sodium edetate etc.), suspending agents (e.g., gum arabic, carmellose, etc.), corrigents (e.g., simple syrup, glucose, etc.), flavors, etc. can be appropriately added. The dosage form for non-oral systemic administration includes injections, suppositories, etc. Injections can be manufactured by using solvents (e.g. water for injection, etc.), stabilizers (e.g., sodium edetate etc.), isotonizing agents (e.g., sodium chloride, glycerol, mannitol, etc.), pH control agents (e.g., hydrochloric acid, citric acid, sodium hydroxide, etc.), suspending agents (e.g., methylcellulose, sodium carboxymethyl cellulose, etc.), and other suitable additives. For the manufacture of suppository, a suppository base (e.g., cacao butter, macrogol, etc) and the like may be appropriately used.

The prophylactic and therapeutic drug of the present invention is useful for the prevention and treatment of neurodegenerative disorders and diseases in mammals (e.g., human, gerbil, rat, mouse, rabbit, cow, pig, dog, cat, and the like). The dosage of the compounds of formula (I-IV), the other compounds described herein or pharmaceutically acceptable salts thereof according to the present invention is dependent on the target disease, clinical state and other conditions of patients, administration route, and other factors. Specific dosages ranges can be determined by routine dose titration experiments. Generally speaking, the objective effect can be achieved in a general dose of 0.001-1000 mg/kg of body weight and preferably 0.01-500 mg/kg of body weight. Unless contrary to the object of the present invention, the neuroprotective agents of the present invention can be used in conjunction with other active ingredients.

I claim:

1. A substantially monodispsere polyglutamate polymer comprising a polyglutamate polymer having a polydispersity (_{w}/Mₙ) of less than about 1.3.

2. The monodisperse polyglutamate of claim 1, wherein the polydispersity (M_{w}/Mₙ) is less than about 1.2.

3. The monodisperse polyglutamate of claim 1, wherein the polydispersity (M_{w}/Mₙ) is about 1.1 or less.

4. A method of inhibiting nitric oxide synthase III (NOS III) in a mammal, which comprises administering to said mammal one or more polyglutamate polymers having a molecular weight of at least 100,000 Daltons in an amount effective to inhibit the activity of NOS III in said mammal.

5. The method of claim 4 wherein the polyglutamate polymer comprises one or more polymers of the formula I (α-polyglutamate) or formula II (γ-polyglutamate) 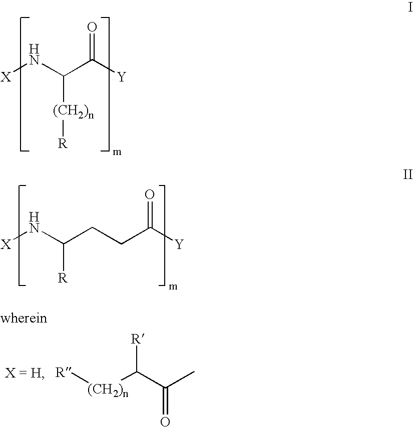
and for formula I: 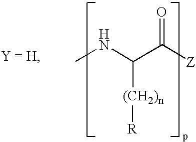
and for formula II: 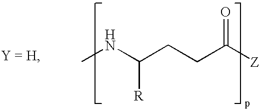
and for formulas I and II: 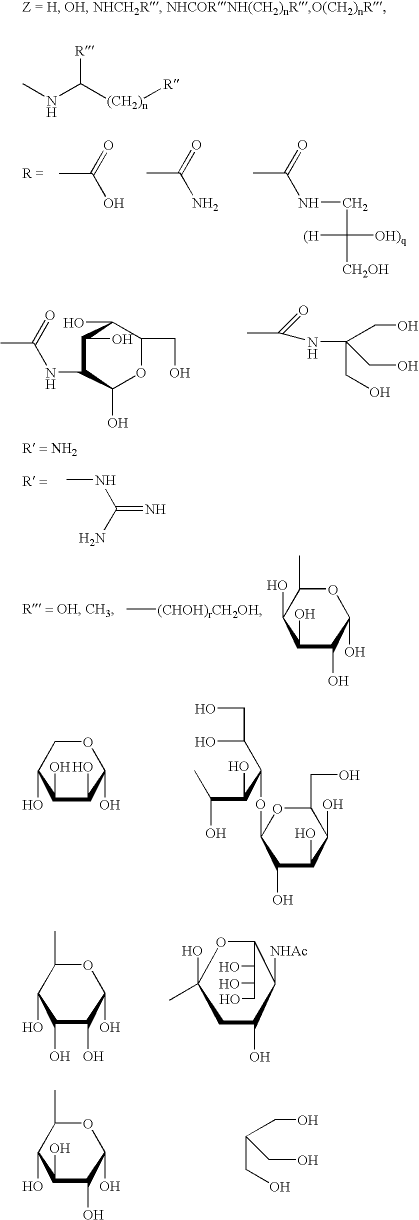
wherein m=1-70,000; n=1-5, p=0-3, q=1-6, r=1-6 and R may also represent the metal salts of carboxylic acids when R=COOH where the metal is an essential metal selected from the group consisting of aluminum, calcium, iron, lithium, manganese, magnesium, copper, selenium, and zirconium.

6. The method of claim 5, wherein the γ-polyglutamate is monodisperse and has a poly dispersity of less than about 1.3.

7. The method of claim 5, wherein the γ-polyglutamate has a molecular weight of about 1,000,000 Daltons.

8. A method of inhibiting nitric oxide synthase III (NOS III) in a mammal, which comprises administering to said mammal one or more arabinogalactan compounds having a 1,3-β-D-galactan backbone with 1,6-β-D-galactobiose, 1,3-β-L-arabinofuranosyl-α-L-arabinofuranose, and α-L-arabinofuranose branch units and a molecular weights of from about 6,000 to about 2,500,000 Daltons in an amount effective to inhibit the activity of NOS III in said mammal.

9. The method of claim 8, wherein the arabinogalactan is a compound of the formula III: 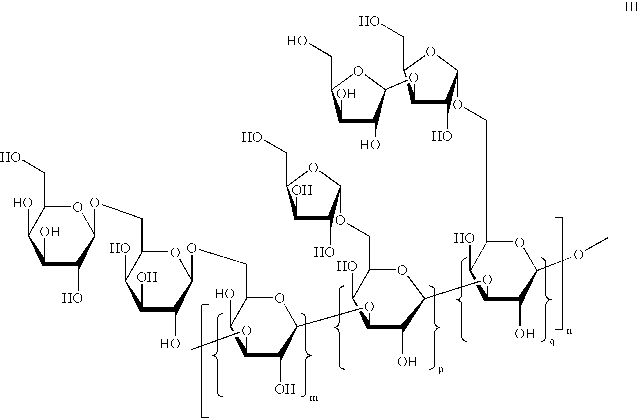
wherein m>q>p and m represents a number between 3 and 300; q represents a number between 2-50; p represents a number between 1 and 20; and n represents a number from 5 to 2,5000.

10. The method of claim 9, wherein m is between 100 and 200; q is between 20-30; p is 3, 4 or 5 and n is about 90.

11. A method of inhibiting nitric oxide synthase III (NOS III) in a mammal, which comprises administering to said mammal one or more acacia (gum arabic) compounds having a 1,3-β-D-galactose backbone with O-6 linked branch units selected from the group consisting of D-galactose, L-arabinofuranose and D-glucuronic and a molecular weights of from about 10,000 to about 2,000,000 Daltons in an amount effective to inhibit the activity of NOS III in said mammal

12. The method of claim 11, wherein the molecular weight of the acacia compounds is about 250,000 Daltons and the compound contains L-rhamnose branches.

13. A method of inhibiting nitric oxide synthase III (NOS III) in a mammal, which comprises administering to said mammal one or more polymers of Formula IV:
Y—(X)ₘ—R (IV)
wherein
Y represents H, (CH₂)ₙ, O(CH₂)ₙ, [(CH₂)₂O]ₙ, N(CH₂)ₙ, alkyl, amino acid, or carbohydrate, (trishydroxymethyl)aminomethane and where n=1-6;
m is 1, 2, 3 or 4;
R represents H, alkyl, amino acid, carbohydrate, halogen, (trishydroxymethyl)aminomethane, polyol or acyl residue; and
X represents benzene, benzidine, S(−)-benzoin, benzophenone, diphenylamine, benzopinacole, cycloheptyl, cyclohexyl, cyclooctyl, cyclopentyl, furan, imidazole, iminostilbine, indazole, indole, indoline, bis(4-aminophenyl-1,4-diisopropylbenzene), phenyl, piperidine, pyridine, pyrrolidone, pyrrole, triazine, triphenylmethane, tryptamine, alkyl-, aryl, and halogen-substituted derivatives thereof, or a substituent of one of the formulas V-XI: 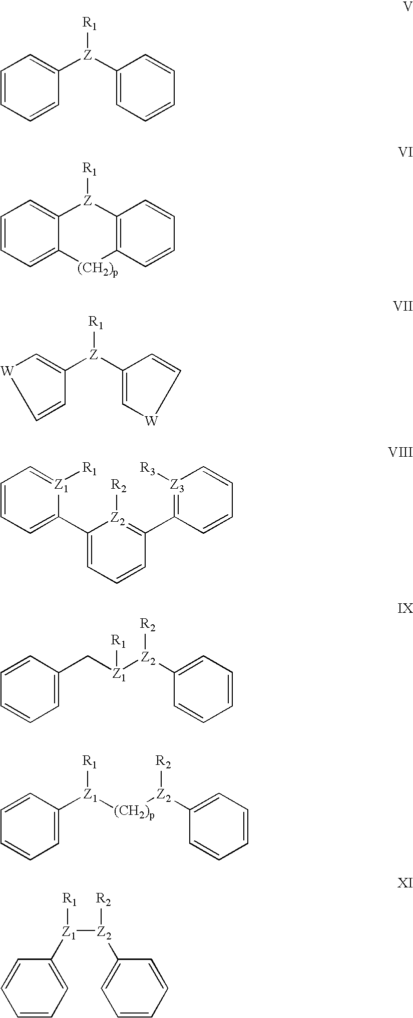
wherein Z, Z₁, Z₂, Z₃ represents (CH₂)ₚ, C═O, CHO, N, NH, C═CHR₁, CH—NH, C═NNH, O, or S;
R₁, R₂, R₃ represent (CH₂)ₖR₄, O(CH₂)ₖR₄, OH, carbohydrate, an amino acid, (trishydroxymethyl)aminomethane, 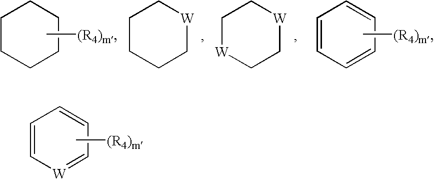
R₄ represents H, alkyl, halogen, N-alkyl, (trishydroxymethyl)aminomethane
W=CH₂, CH₂R₅, CH, CHR₅, N(R₅)ⱼ, N, O, S, C═O
R₅=H, F, OH, alkyl, aryl and
where j=1-2, k=1-6, m′=1-5, p=1-10, q=1-6.

14. A method of inhibiting nitric oxide synthase III (NOS III) in a mammal, which comprises administering to said mammal one or more polymeric metal complexes of Formula XII
P—[M] (XII)
wherein P is an anionic polymer, selected from the group consisting of a polysaccharide, oligosaccharide, or a polypeptide polymer; and M is a neuroprotective metal ion selected from the group consisting of aluminum, calcium, copper, lithium, magnesium, manganese, selenium, iron and zirconium and pharmaceutically acceptable salts thereof in an amount effective to inhibit the activity of NOS III in said mammal.

15. The method of claim 14, wherein the polymeric metal complex is a polysaccharide iron complex or a poly(glutamic acid) iron complex.

16. A method of inhibiting nitric oxide synthase III (NOS III) in a mammal, which comprises administering to said mammal an effective NOS III inhibiting amount of one or more tripeptides selected from the group consisting of arginine-glutamate-arginine, arginine-asparagine-arginine, lysine-glutamate-arginine, arginine-glutamate-lysine, ornithine-glutamate-arginine, arginine-glutamate-ornithine, citrulline-glutamate-arginine, arginine-glutamate-citrulline, N-acetyl-arginine-glutamate-arginine, arginine-glutamate-arginine-NH₂, arginine-glutamate-arginine-OCH₃, D-arginine-L-glutamate-arginine, L-arginine-D-glutamate-arginine and L-arginine-L-glutamate-arginine.

17. A method of inhibiting nitric oxide synthase III (NOS III) in a mammal, which comprises administering to said mammal an effective NOS III inhibiting amount of one or more compounds selected from the group consisting of p-Aminoclonidine; Aminopentamide; Amperozide; Atenolol; Atropine; Bepridil; Bietanautine 1,2,3,6-Tetrahydro-1,3-dimethyl-2,6-dioxo-7H-purine-7-acetic acid compound with 2-(diphenylmethoxy)-N,N-dimethylamine (2:1); 1-(2-(Bis-(4-fluorophenyl)methoxy)ethyl)-4-(3-phenylpropyl)-piperazine); 1-(2-(Bis-(4-fluorophenyl)methoxy)ethyl)-4-(3-phenyl-2-propenyl)-piperazine); Bromocriptine; Buaverine; Bulan; Buspirone HCl; Butyl-N-ethyl-2-(1-naphthyloxy); Clonidine; Ephedrine; Ethaneamine; Calmidazolium chloride; Carbamazepine; Cetinzne; Cetrizine; Chicago Sky Blue 6B; 1-(2-Chlorobenzoyl)piperazine-2,3-dicarboxylic acid, Na; 2-Chloro-11-(4-methylpiperazino)dibenzy[b,f]oxepin]; 3α[(4-Chlorophenyl)phenylmethoxy]tropane; Cinnarizine; Ciprofloxacin; Clomipramine; Cyproheptadine; Deoprenilamine; Deofenine; Deprenyl; N-Desmethylclozapine; Diazepam; (2-(3,4-Dichlorophenyl)-N-methyl-[(1S)-1-phenyl-2-(1-pyrrolidoninyl)ethy](acetamide); (RS-[3-[1-[((3,4-Dichlorophenyl)acetyl]methylamino]-2-(1-pyrrolidoninyl)ethyl]phenoxy] acetic acid); N,N-Diethyl-2[4-phenylmethyl) phenoxy]ethanmine); 4,4′-Difluoro-3α-(diphenylmethoxy)tropane hydrochloride; 1,1-Dimethyl-4-diphenylacetoxypiperinium iodide (4-DAMP); Dimepheptanol; Diphenhydramine HCl; Diphenidol; Diphenolic acid; (1-(4,4-Diphenyl-3-butenyl)-3-piperidinecarboxylic acid); [1-(2-Diphenylmethoxyethyl)-4-(3-phenylpropyl)piperazine); [1-(2-Diphenylmethoxyethyl)-4-(3-phenyl-2-propenyl)-piperazine); Diphenylpyraline; Dipipaone; Doxepin; Doxylamine; Droloxifene; Edrophenoium chloride; Emepronium bromide; Ergonamine; Ergotamine; Etastine; Etifelmin; Etodioxizine; Ethylbenzhydramine; Ethylbentropine; Felodidine; Fendiline; Feniprane; Fenoprofen; Fenpiverinium bromide; Fexofenadine; Fludpirilene; Flufenamic acid; Flunarizine; Fluoxetine; Flupentixol dihydrochloride; Fluphenazine-N-2-chloroethane; Flusipirilene; Galanthamine hydrobromide; Genistein; N-[3-(1-Hexahydroazapinyl)propyl]-a-cyclohexylbenzeneacetamide; Ifenprodil; Imipramine; Ipatropium bromide; Isomethadol; Isomethadone; Ketanserin tartrate; Ketotifen fumerate; Levomethadyl acetate; Lidoflazine; Lorazepam; Lupinifolin; Lupinifolinol; Meperidine; Methadone; Methadyl acetate; Methiothepin maleate; 2-[2-(4-(2-Methoyphenyl)piperazine-1-yl)ethyl]-4,4-dimethyl-1,3-(2H,4H)-isoquinolindione HCl; d-Methylphenidate; Mianserin HCl; Naftopidil dihydrochloride; Neostigmine; Noracymethadol; Normethadone; Norpipanone; Oxatomide; Oxazepam; Penfluridol; Pentacynium bis(methylsulfate); Pergolide; Phenadoxone; Phenoxybenzamine; Phentolamine; α-Phenyl-1-(2-phenethyl)-4-piperidinemethanol; Physostigmine; Pimozide; Piocarpine; Pirenzepine dihydrochloride; Propanolol; Pyridostigmine bromide; Rilmenidine; Rimcazole dihydrochloride; Ritanserin; Robinetin; 12-epi-Scalaradial; Scopilamine; Selegilline; Sennoside; Sertraline; Sulpiride; Tacrine; Tamoxifen; Tamoxifen, 4-hydroxy/3-hydroxy; N,N,N′,N′-Tetrakis-[(2-pyridylmethyl)ethylenediamine]; Thiothixene HCl; Trifluoroperazine; Trihexyphenidyl hydrochloride; 3-Tropanyl-3,5-dichlorobenzoate; Yohimbine; and Zimelidine dihydrochloride.
